(19) United States
(12) Patent Application Publication
(10) Pub. No.: US 2022/0162626 A1
(43) Pub. Date: May 26, 2022

(54) Modulation of Transgene Expression

(71) Applicant: Syngenta Crop Protection AG, Basel (CH)

(72) Inventors: Julie Leonard Green, Research Triangle Park, NC (US); Timothy Joseph KELLIHER, Research Triangle Park, NC (US)

(73) Assignee: Syngenta Crop Protection AG, Basel (CH)

(21) Appl. No.: 17/441,509

(22) PCT Filed: Mar. 26, 2020
(86) PCT No.: PCT/US2020/024823

§ 371 Date: Sep. 21, 2021

Related U.S. Application Data
(60) Provisional application No. 62/825,107, filed on Mar. 28, 2019.

(51) Int. Cl. C12N 15/82

(52) U.S. Cl. CPC C12N 15/8218; C12N 15/8286

(57) ABSTRACT

Compositions and methods of modulating, for example reducing, recombinant insecticidal protein expression in male reproductive cells and/or tissues of transgenic plants are disclosed. In particular, novel recombinant DNA constructs useful in such methods, as well as transgenic plants, cells, and seeds containing such recombinant DNA constructs are provided. The recombinant DNA constructs and the transgenic plants, cells, and seeds containing such constructs provide a greatly improved way to minimize any potential risks that may be associated with expression of insecticidal proteins in male reproductive tissues.

### SEQUENCE LISTING

A Sequence Listing in ASCII text format, submitted under 37 C.F.R. § 1.821, entitled “81814-US-L-ORG-P-1_ST25.txt”, 701 kilobytes in size, generated on Mar. 28, 2019 and filed via EFS-Web is provided in lieu of a paper copy. This Sequence Listing is hereby incorporated by reference into the specification for its disclosures.

### FIELD OF THE INVENTION

The present invention discloses molecular constructs and methods for the control of transgene expression, for example, transgene suppression in plants or suppressing expression of a target RNA in a specific cell or tissue. Also disclosed are transgenic eukaryotes, including transgenic plant cells, plants, and seeds, whose genome includes molecular constructs for controlling expression of an exogenous gene.

### BACKGROUND

Transgenic crops consist of increasingly complex genetic modifications including multiple transgenes that confer different traits, also called “gene stacks” or “trait stacks.” For example, many transgenic corn products currently on the market contain within the same plant multiple genes encoding insecticidal proteins for controlling a broad spectrum of insect pests, multiple genes encoding proteins that confer on the plant tolerance to a wide spectrum of chemical herbicides and multiple genes encoding proteins that are used as selectable markers during the plant transformation process. Many of the transgenic proteins used to control insect pests, for example the crystal endotoxins from Bacillus thuringiensis (called Cry proteins) are active against lepidopteran or coleopteran insect pests. Examples of lepidopteran-active Cry proteins include Cry1A, Cry1B, Cry1C, Cry1D, Cry1E, Cry1F and Cry9. Examples of coleopteran-active Cry proteins include, Cry3A, Cry3B, Cry3C, Cry8, the binary Cry23-Cry37 and the binary Cry34-Cry35. Most individual Cry proteins are biologically active against a narrow spectrum of insect species within a given insect order. Even with this narrow spectrum of activity, certain Cry proteins may have low to moderate activity against certain non-pest species in the same order of insects as the target pest insects. For example, Hellmich et al. (2001) Proc. Natl. Aca. Sci. 98:11925-11930, found that certain purified Cry proteins that are active against a lepidopteran pest, e.g. European corn borer (Ostrinia nubilalis), also have some activity against the first instar of the non-pest lepidopteran insect, monarch butterfly (Danaus plexippus). However, later larval instars D. plexippus were far less susceptible.

Currently, expression of most transgenes encoding insecticidal proteins in commercial transgenic crops is driven by constitutive promoters, i.e. promoters that are functional throughout the plant in all or a majority of tissue types, including pollen, throughout the entire growth cycle of the plant. Since plant pollen may be a food source for some non-pest insect species or it is hypothesized that plant pollen may be carried by wind to deposit on non-pest insect host plants, there is some concern within regulatory agencies that regulate transgenic crop commercialization that high levels of expression of certain insecticidal proteins, e.g. certain Cry proteins, in pollen may have adverse effects on localized populations of non-pest insects. In addition, it has been observed that expression of certain insecticidal proteins in pollen has adverse effects on the transgenic plant's male fertility. For example, high levels of a Vip3 insecticidal protein expressed in corn pollen may cause a decrease in male fertility or complete sterility in certain inbred genetic backgrounds (U.S. Pat. No. 10,214,784; herein incorporated by reference). Therefore, it would be beneficial to modulate the expression of insecticidal proteins in transgenic plants, for example, to have high levels of expression in vegetative tissues, e.g. leaf tissue, where a majority of pest insects initially feed, but have reduced expression in pollen, a plant tissue that some non-pest insects may feed upon.

Most research directed at targeting plant transgene expression in a temporal and/or spatial manner has focused on the use of promoters, from viral and plant constitutive promoters, to tissue- and cell-specific promoters, to inducible and synthetic promoters. Promoter selection has become increasingly important for successful gene transfer and expression of transgenes in plants. However, quite frequently, the spatial and temporal functionality of a promoter in its natural setting, e.g. a wild-type non-transgenic plant, does not predict the spatial and temporal functionality when used in a nucleic acid construct to drive expression of a heterologous transgene. For example, a promoter that has root-specific functionality in wild-type (non-transgenic) corn plants, may function as a constitutive promoter or a root-preferred only promoter, i.e. expresses more highly in roots but also has leaky expression in other tissues such as vegetative tissues, when it is used to drive a heterologous transgene in transgenic corn. Therefore, alternatives to using promoters or in addition to promoters to control transgene expression in plants is needed to modulate, e.g. suppress or increase, expression in certain tissues of transgenic crops, particularly in male reproductive tissues like pollen and/or tapetum. Ideally, the alternative methods and compositions would modulate, for example suppress, the expression of a transgene that encodes an insecticidal protein in male reproductive cells or tissues, regardless of what promoter is used to drive expression of the transgene in the transgenic plant.

Mechanisms that suppress the expression of specific cellular genes, viruses or mobile genetic elements (such as transposons and retro-elements) are critical for normal cellular function in a variety of eukaryotes. A number of related processes, discovered independently in plants (Matzke et al., Curr. Opin. Genet. Dev. 11:221-227, 2001), animals (Fire et al., Nature, 391:806-811, 1998) and fungi (Cogoni, Annu. Rev. Microbiol. 55:381-406, 2001), result in the RNA-directed inhibition of gene expression (also known as RNA silencing). Each of these processes is triggered by molecules containing double-stranded RNA (dsRNA) structure, such as transcripts containing inverted repeats or double-stranded RNA intermediates formed during RNA virus replication. Non-dsRNAs, also referred to as aberrant RNAs, may also function as initiators of RNA silencing. Such aberrant RNAs may be converted into dsRNAs by silencing-associated RNA-dependent RNA polymerases (RDRs), which have been identified in plants, fungi and the nematode C. elegans (Tuschl, Chem Biochem, 2:239-245, 2001).

Two major classes of small RNAs have been characterized: short interfering RNAs (siRNAs) and microRNAs (miRNAs). The primary transcripts that eventually form miRNAs are transcribed from non-protein-coding miRNA genes. These transcripts form hairpin structures that are then processed by Dicer (or by Dicer-like activities in plants) to yield small RNA duplexes containing 2-base overhangs at each 3′ end. The mature single-stranded miRNA approximately 20-22 nucleotides in length forms by dissociation of the two strands in the duplex, and is selectively incorporated into the RNA-Induced Silencing Complex, or RISC (Zamore, Science, 296:1265-1269, 2002; Tang et al., Genes Dev., 17:49-63, 2003; Xie et al., Curr. Biol. 13:784-789, 2003).

siRNAs are similar in chemical structure to miRNAs, however siRNAs are generated by the cleavage of relatively long double-stranded RNA molecules by Dicer or DCL enzymes (Zamore, Science, 296:1265-1269, 2002; Bernstein et al., Nature, 409:363-366, 2001). In animals and plants, siRNAs are assembled into RISC and guide the sequence specific ribonucleolytic activity of RISC, thereby resulting in the cleavage of mRNAs, viral RNAs or other RNA target molecules in the cytoplasm. In the nucleus, siRNAs also guide heterochromatin-associated histone and DNA methylation, resulting in transcriptional silencing of individual genes or large chromatin domains.

MicroRNAs in plants and animals function as posttranscriptional regulators of genes involved in a wide range of cellular processes (Bartel, Cell 116:281-297, 2004; He & Hannon, Nat Rev Genet. 5:522-531, 2004). In the plant Arabidopsis thaliana, miRNAs regulate mRNAs encoding at least twelve families of transcription factors, several miRNA metabolic factors, and proteins involved in stress responses, metabolism, and hormone signaling (Jones-Rhoades & Bartel, Mol Cell 14:787-799, 2004; Kasschau et al., Dev Cell 4:205-217, 2003; Llave et al., Science 297:2053-2056, 2002b; Vazquez et al., Curr Biol 14:346-351, 2004a; Xie et al., Curr Biol 13:784-789, 2003). Plant miRNAs target a disproportionately high number of genes with functions in developmental processes, including developmental timing, control of cell proliferation, meristem cell function, and patterning. Global disruption of miRNA biogenesis or function, or specific disruption of miRNA-target interactions, can result in severe developmental abnormalities (Achard et al., Development 131:3357-3365, 2004; Chen, Science 303:2022-2025, 2004; Emery et al., Curr Biol 13:1768-1774, 2003; Juarez et al., Nature 428:84-88, 2004; Kidner & Martienssen, Nature 428:81-84, 2004; Laufs et al., Development 131:4311-4322, 2004; Mallory et al., Curr Biol 14:1035-1046, 2004; Palatnik et al., Nature 425:257-263, 2003; Tang et al., Genes & Dev 17:49-63 2003; Vaucheret et al., Genes Dev 18:1187-1197, 2004), indicating that miRNA-based regulation is crucial for normal growth and development. This idea is reinforced by the conservation of most miRNAs and their corresponding targets through significant evolutionary time (Bartel, Cell 116:281-297, 2004). MicroRNAs have been identified by direct cloning methods and computational prediction strategies (Jones-Rhoades & Bartel, Mol Cell 14:787-799, 2004; Llave et al., Plant Cell 14:1605-1619, 2000a; Park et al., Curr Biol 12:1484-1495, 2002; Reinhart et al., Genes Dev 16:1616-1626, 2002; Sunkar & Zhu, Plant Cell 16:2001-2019, 2004).

Plant miRNAs usually contain near-perfect complementarity with target sites, which are found most commonly in protein-coding regions of the genome. As a result, most (but not all) plant miRNAs function to guide cleavage of targets through a mechanism similar to the siRNA-guided mechanism associated with RNAi (Jones-Rhoades & Bartel, Mol Cell 14:787-799, 2004; Kasschau et al., Dev Cell 4:205-217, 2003; Llave et al., Science 297:2053-2056, 2002; Tang et al., Genes & Dev 17:49-63 2003). In contrast, animal miRNAs contain relatively low levels of complementarity to their target sites, which are most commonly found in multiple copies within 3′ untranslated regions of the target transcript (Lewis et al., Cell 115:787-798, 2003; Rajewsky & Socci, Dev Biol 267:529-535, 2004; Stark et al., PLoS Biol 1:E60, 2003). Most animal miRNAs do not guide cleavage, but rather function to repress expression at the translational or co-translational level (Ambros, Cell 113:673-676, 2003; He & Hannon, Nat Rev Genet. 5:522-531, 2004). At least some plant miRNAs may also function as translational repressors (Aukerman & Sakai, Plant Cell 15:2730-2741, 2003; Chen, Science 303:2022-2025, 2004). Translation repression is not an inherent activity of animal miRNAs, as miRNAs will guide cleavage if presented with a target containing high levels of complementarity (Doench et al., Genes Dev 17:438-442, 2003; Hutvagner & Zamore, Science 297:2056-2060, 2002; Yekta et al., Science 304:594-596, 2004; Zeng et al., Proc Natl Acad Sci USA 100:9779-9784, 2003).

MicroRNAs form through nucleolytic maturation of genetically defined RNA precursors that adopt imperfect, self-complementary fold-back structures. Processing yields a duplex intermediate (miRNA/miRNA*) that ultimately provides the miRNA strand to the effector complex, termed RISC (Khvorova et al., Cell 115:209-216, 2003; Schwarz et al., Cell 115:199-208, 2003). Plants contain four DICER-LIKE (DCL) proteins, one of which (DCL1) is necessary for maturation of most or all miRNA precursors (Kurihara & Watanabe, Proc Natl Acad Sci USA 101:12753-12758, 2004; Park et al., Curr Biol 12:1484-1495, 2002; Reinhart et al., Genes Dev 16:1616-1626, 2002; Schauer et al., Trends Plant Sci 7:487-491, 2002). The DCL1 protein contains an RNA helicase and two RNaseIII-like domains, a central PAZ domain and C-terminal dsRNA binding motifs. Animal miRNA precursor processing requires Drosha, another RNaseIII domain protein, and Dicer in sequential nucleolytic steps (Lee et al., Nature 425:415-419, 2003). HEN1 participates in miRNA biogenesis or stability in plants via a 3′ methylase activity (Boutet et al., Curr Biol 13:843-848, 2003; Park et al., Curr Biol 12:1484-1495, 2002). The dsRNA-binding HYL1 protein is necessary for miRNA biogenesis in cooperation with DCL1 and HEN1 in the nucleus. Based on sequence similarity, HYL1 has been suggested to function like animal R2D2, which is required post-processing during RISC assembly (Han et al., Proc Natl Acad Sci USA 101:1093-1098, 2004; Liu et al., Science 301:1921-1925, 2003; Pham et al., Cell 117:83-94, 2004; Tomari et al., Science 306:1377-1380, 2004; Vazquez et al., Curr Biol 14:346-351, 2004a). In animals, Exportin-5 (Exp5) regulates the transport of pre-miRNAs from the nucleus to the cytoplasm by a Ran-GTP-dependent mechanism (Bohnsack et al., RNA 10:185-191, 2004; Lund et al., Science 303:95-98, 2003; Yi et al., Genes Dev 17:3011-3016, 2003). In Arabidopsis, HST may provide a related function to transport miRNA intermediates to the cytoplasm (Bollman et al., Development 130:1493-1504, 2003). Active miRNA-containing RISC complexes in plants almost certainly contain one or more ARGONAUTE proteins, such as AGO1 (Fagard et al., Proc Natl Acad Sci USA 97:11650-11654, 2000; Vaucheret et al., Genes Dev 18:1187-1197, 2004). Argonaute proteins in animals were shown recently to provide the catalytic activity for target cleavage (Liu et al., Science 305:1437-1441, 2004; Meister et al., Mol Cell 15:185-197, 2004).

In addition to miRNAs, plants also produce diverse sets of endogenous 21-25 nucleotide small RNAs. Most of these differ from miRNAs in that they arise from double-stranded RNA (rather than imperfect fold-back structures), in some cases generated by the activity of RNA-DEPENDENT RNA POLYMERASEs (RDRs). Arabidopsis DCL2, DCL3, DCL4, RDR1, RDR2 and RDR6 have known roles in siRNA biogenesis (Dalmay et al., Cell 101:543-553, 2000; Mourrain et al., Cell 101:533-542, 2000; Peragine et al., Genes & Dev 18:2369-2379, 2004; Vazquez et al., Mol Cell 16:69-79, 2004b; Xie et al., PLoS Biol 2:642-652, 2004; Yu et al., Mol Plant Microbe Interact 16:206-216, 2003). For example, DCL3 and RDR2 cooperate in the heterochromatin-associated RNAi pathway, resulting in .about.24-nucleotide siRNAs from various retro-elements and transposons, 5S rDNA loci, endogenous direct and inverted repeats, and transgenes containing direct repeats (Xie et al., PLoS Biol 2:642-652, 2004; Zilberman et al., Science 299:716-719, 2003). RDR6 functions in posttranscriptional RNAi of sense transgenes, some viruses, and specific endogenous mRNAs that are targeted by trans-acting siRNAs (ta-siRNAs) (Dalmay et al., Cell 101:543-553, 2000; Mourrain et al., Cell 101:533-542, 2000; Peragine et al., Genes & Dev 18:2369-2379, 2004; Vazquez et al., Mol Cell 16:69-79, 2004b; Yu et al., Mol Plant Microbe Interact 16:206-216, 2003). Ta-siRNAs arise from transcripts that are recognized by RDR6, in cooperation with SGS3, as a substrate to form dsRNA. The dsRNA is processed accurately in 21-nucleotide steps by DCL1 to yield a set of “phased” ta-siRNAs. These ta-siRNAs interact with target mRNAs to guide cleavage by the same mechanism as do plant miRNAs (Peragine et al., Genes & Dev 18:2369-2379, 2004; Vazquez et al., Mol Cell 16:69-79, 2004).

In view of the above-described problems with modulating transgene expression in transgenic plants, the present invention provides novel DNA constructs comprising novel miRNA elements as well as methods of using the miRNA elements and constructs to modulate expression of transgenes encoding insecticidal proteins in male reproductive tissues of transgenic plants. Such compositions and methods take advantage of endogenous microRNAs that are functional in tissues in which modulation of transgene expression is desired.

### SUMMARY

The invention relates generally to methods of modulating, for example reducing, recombinant insecticidal protein expression in male reproductive cells and/or tissues of transgenic plants, recombinant DNA constructs useful in such methods, as well as transgenic plants, cells, and seeds containing such recombinant DNA constructs. The recombinant DNA constructs and the transgenic plants, cells, and seeds containing such constructs provide a greatly improved way to minimize any potential risks to non-pest insect species associated with expression of insecticidal proteins in pollen or for mitigating the impact that expression of insecticidal proteins in pollen and/or tapetum may have on male fertility in transgenic plants.

In one aspect, the invention provides a recombinant DNA construct that includes a protein-coding sequence encoding a recombinant protein, for example an insecticidal protein, and a DNA that transcribes to a microRNA (miRNA) element capable of initiating binding with an endogenous plant miRNA that is male tissue-specific or male tissue-preferred comprising, for example, a miRNA initiator sequence, operably linked to the protein-coding sequence. In one embodiment, the miRNA element is included within the 3′ untranslated region of the protein-coding sequence. In another embodiment, the miRNA element is included within the 5′ untranslated region of the protein-coding sequence. In another embodiment, the miRNA element is included within the protein-coding sequence, for example between the start codon and the stop codon of the protein coding sequence. In another embodiment, the miRNA element is located between the protein-coding sequence and a polyadenylation sequence which is part of a 3′ untranslated region. In another embodiment, the miRNA element includes at least one miRNA initiator sequence. In another embodiment, the expression of a miRNA initiator sequence in a transgenic corn plant reduces the expression of an insecticidal protein of the invention in male reproductive tissue of the transgenic corn plant compared to non-male reproductive tissue in the transgenic corn, such leaf tissue. In other embodiments, expression of a miRNA initiator sequence of the invention in a transgenic corn plant increases the expression of an insecticidal protein of the invention in male reproductive tissue, such as pollen, of the transgenic corn plant compared to non-male reproductive tissue in the transgenic corn, such leaf tissue. In another embodiment, the miRNA element includes at least one sequence that encodes a miRNA initiator sequence selected from the group consisting of SEQ ID NO: 41-46 or SEQ ID NO: 98-101, which encode a miRNA initiator sequence selected from the group consisting of SEQ ID NO: 29-34 or SEQ ID NO: 94-97, respectively. In another embodiment, the miRNA element comprises a target insertion sequence comprising a miRNA initiator sequence and a synthetic nucleotide sequence flanking the 5′ and/or the 3′ end of the initiator sequence. In another embodiment, the target insertion sequence is selected from the group consisting of SEQ ID NO: 10-18 or SEQ ID NO: 79-83. In another embodiment, the expression of the recombinant insecticidal protein in a transgenic plant confers tolerance in at least vegetative tissues to feeding damage from insect pests. In another embodiment, the recombinant insecticidal protein is an insecticidal Cry protein or a vegetative insecticidal protein (Vip). In another embodiment, the insecticidal protein is a Cry1A protein or a Vip3A protein. In another embodiment the insecticidal protein is a Cry1Ab protein or a Vip3Aa protein. In another embodiment, the insecticidal protein comprises an amino acid sequence selected from the group consisting of SEQ ID NO: 61-66 or SEQ ID NO: 115-117.

Another aspect of the invention provides an expression cassette comprising a heterologous promoter operably linked to a recombinant insecticidal protein coding-sequence operably linked to a DNA sequence encoding a microRNA (miRNA) element of the invention that is male tissue-specific or male tissue-preferred in a corn plant. In one embodiment, the expression cassette comprises a sequence selected from the group consisting SEQ ID NO: 20-28 or SEQ ID NO: 85-93.

Another aspect of the invention provides a recombinant vector comprising a DNA construct of the invention. In one embodiment, the recombinant vector comprises a sequence selected from the group consisting of SEQ ID NO: 48-59 or SEQ ID NO: 105-113.

Another aspect of the invention provides a transgenic corn (maize; Zea mays) plant comprising a DNA construct and/or an expression cassette and/or a recombinant vector of the invention. In one embodiment, the invention provides transgenic seed, progeny or a plant part of the transgenic corn plant of the invention, wherein the transgenic seed, progeny or plant part comprises a DNA construct, expression cassette or recombinant vector of the invention.

Another aspect of the invention provides a method of making a DNA construct comprising identifying an endogenous male tissue-specific or male tissue-preferred corn microRNA (miRNA); constructing a miRNA element that encodes at least one miRNA initiator sequence that is recognized by the corn miRNA and operably linking the miRNA element to an insecticidal protein-coding sequence. In one embodiment, the male tissue-specific miRNA or male tissue-preferred miRNA is pollen-specific or pollen-preferred or tapetum-specific or tapetum preferred. In another embodiment, the male tissue-specific miRNA element or male tissue-preferred miRNA element is pollen-specific or pollen-preferred or tapetum-specific or tapetum preferred. In another embodiment, the miRNA element comprises a sequence selected from the group consisting of SEQ ID NO: 10-18 or SEQ ID NO: 79-83. In another embodiment, the miRNA element includes at least one sequence that encodes a miRNA initiator sequence selected from the group consisting of SEQ ID NO: 41-46 or SEQ ID NO: 98-101, which encode a miRNA initiator sequence selected from the group consisting of SEQ ID NO: 29-34 or SEQ ID NO: 94-97, respectively.

In a further aspect, the invention also provides a method of modulating and/or selectively reducing the expression of a recombinant insecticidal protein in a male reproductive cell or tissue of a transgenic corn plant by expressing in the transgenic corn plant a recombinant DNA construct comprising an insecticidal protein-coding sequence operably linked to a DNA sequence including an miRNA element of the invention. In one embodiment, the miRNA element includes at least one miRNA initiator sequence. In another embodiment, the miRNA element includes a target insertion sequence comprising a miRNA initiator sequence and a synthetic nucleotide sequence flanking the 5′ and/or the 3′ end of the initiator sequence. In another embodiment, the male reproductive tissue is pollen and/or tapetum. In another embodiment, the miRNA element includes at least one miRNA initiator sequence selected from the group consisting of SEQ ID NO: 41-46 or SEQ ID NO: 98-101, which encode a miRNA initiator sequence selected from the group consisting of SEQ ID NO: 29-34 or SEQ ID NO: 94-97, respectively. In another embodiment, the target insertion sequence is selected from the group consisting of SEQ ID NO: 10-18 or SEQ ID NO: 79-83. In another embodiment, the expression of the recombinant insecticidal protein in a transgenic corn plant confers tolerance in at least vegetative tissues to feeding damage from insect pests. In another embodiment, the recombinant insecticidal protein is an insecticidal Cry protein or a vegetative insecticidal protein (Vip). In another embodiment, the insecticidal protein is a Cry1A protein or a Vip3A protein. In another embodiment the insecticidal protein is a Cry1Ab protein or a Vip3Aa protein. In another embodiment, the insecticidal protein comprises an amino acid sequence selected from the group consisting of SEQ ID NO: 61-66 or SEQ ID NO: 115-117.

The invention also provides a method of increasing male fertility in a transgenic corn plant expressing a recombinant insecticidal protein that causes reduce male fertility or complete male sterility, including the step of inserting into a genome of a corn plant a recombinant DNA construct of the invention comprising a male sterility inducing insecticidal protein-coding sequence, operably linked to a DNA sequence including an miRNA element that modulates or selectively reduces the expression of the male sterility inducing insecticidal protein in a male reproductive cell or tissue, wherein the transgenic corn plant comprising the DNA construct has increased male fertility compared to a control plant not comprising the DNA construct. In one embodiment, the male sterility inducing protein is a Vip3 insecticidal protein. In another embodiment, the Vip3 protein is a Vip3Aa protein. In another embodiment, the Vip3 protein comprises an amino acid sequence selected from the group consisting of SEQ ID NO: 114-121.

The invention also provides a method for producing a Vip3-expressing corn plant having increased male fertility compared to a Vip3-expressing maize plant that has reduced male fertility or is male infertile, including: (a) providing a first Vip3-expressing corn plant comprising a recombinant DNA construct of the invention comprising a Vip3-coding sequence operably linked to a DNA sequence including an miRNA element that modulates or selectively reduces the expression of the Vip3 protein in a male reproductive cell or tissue; b) introgressing the recombinant DNA construct of (a) into a second corn plant; and c) selecting a Vip3-expressing corn plant comprising the recombinant DNA construct, wherein the miRNA element reduces the level of the Vip3 protein in a male reproductive tissue, thereby producing a Vip3-expressing corn plant with increased male fertility compared to a Vip3-expressing maize plant without the recombinant DNA construct.

The invention also provides a method of reducing the impact of pollen from a transgenic corn plant expressing at least one insecticidal protein on a non-target insect species susceptible to the insecticidal protein including the step of inserting into a genome of a corn plant a recombinant DNA construct of the invention comprising an insecticidal protein-coding sequence operably linked to a DNA sequence including an miRNA element that modulates or selectively reduces the expression of the insecticidal protein in pollen, wherein the pollen from the transgenic corn plant has decreased levels of the insecticidal protein compared to the vegetative tissue of the transgenic corn plant or to pollen from a control plant not comprising the DNA construct.

In another aspect, the invention provides provides a recombinant insecticidal protein that is active against a lepidopteran insect, wherein the insecticidal protein comprises an amino acid sequence that is encoded by a male tissue-specific or male tissue-preferred miRNA element, and wherein the miRNA element encodes at least one miRNA initiator sequence. In one embodiment, the insecticidal protein is a Cry1 protein or a Vip3 protein. In another embodiment, the Cry1 protein is a Cry1A protein or the Vip3 protein is a Vip3A protein. In another embodiment, the Cry1A protein is a Cry1Ab protein and the Vip3A protein is a Vip3Aa protein. In another embodiment, the amino acid sequence is selected from the group consisting of SEQ ID NO: 61-66 or SEQ ID NO: 115-117. In another embodiment, the recombinant insecticidal protein is active against a lepidopteran pest that is a European corn borer or a corn earworm. In another embodiment, the male tissue is pollen or tapetum. In another embodiment, the miRNA element comprises a sequence selected from the group consisting of SEQ ID NO: 10-18 or SEQ ID NO: 74-78. In another embodiment, the miRNA initiator sequence is selected from the group consisting of SEQ ID NO: 29-34 or SEQ ID NO: 94-97.

In another aspect, the invention provides a synthetic polynucleotide encoding a recombinant insecticidal protein of the invention. In one embodiment, the synthetic polynucleotide comprises a sequence selected from the group consisting of SEQ ID NO: 68-73 or SEQ ID NO: 118-121.

Other aspects and advantages of the present invention will become apparent to those skilled in the art from a study of the following description of the invention and non-limiting examples.

### BRIEF DESCRIPTION OF THE DRAWINGS

FIG. 1 depicts ELISA results of T0 plants as described in Example 6. Each bar represents the expression level of a Cry1Ab insecticidal protein as ng Cry1Ab/mg TSP (total soluble protein) in pollen (solid bar) and leaf (open bar). Error bars are ±Standard Error (SE)

### BRIEF DESCRIPTION OF THE SEQUENCES IN THE SEQUENCE LISTING

SEQ ID NO: 1 is an RNA sequence of a 24373miR159h-3p target insertion element.

SEQ ID NO: 2 is an RNA sequence of a 24374miR156i-3p target insertion element.

SEQ ID NO: 3 is an RNA sequence of a 24375miR171i-5p target insertion element.

SEQ ID NO: 4 is an RNA sequence of a 24376miR396b-3p target insertion element.

SEQ ID NO: 5 is an RNA sequence of a 24377miR396b-3p target insertion element.

SEQ ID NO: 6 is an RNA sequence of a 24379miR159h-3p target insertion element.

SEQ ID NO: 7 is an RNA sequence of a 24380miR156i-3p target insertion element.

SEQ ID NO: 8 is an RNA sequence of a 24381miR171i-5p target insertion element.

SEQ ID NO: 9 is an RNA sequence of a 24383miR396b-3p target insertion element.

SEQ ID NOs: 10 is a DNA sequence of a 24373miR159h-3p target insertion sequence.

SEQ ID NO: 11 is a DNA sequence of a 24374miR156i-3p target insertion sequence.

SEQ ID NO: 12 is a DNA sequence of a 24375miR171i-5p target insertion sequence.

SEQ ID NO: 13 is a DNA sequence of a 24376miR396b-3p target insertion sequence.

SEQ ID NO: 14 is a DNA sequence of a 24377miR396b-3p target insertion sequence.

SEQ ID NO: 15 is a DNA sequence of a 24379miR159h-3p target insertion sequence.

SEQ ID NO: 16 is a DNA sequence of a 24380miR156i-3p target insertion sequence.

SEQ ID NO: 17 is a DNA sequence of a 24381miR171i-5p target insertion sequence.

SEQ ID NO: 18 is a DNA sequence of a 24383miR396b-3p target insertion sequence.

SEQ ID NO: 19 is a 24366 cry1Ab expression cassette.

SEQ ID NO: 20 is a 24373 cry1Ab expression cassette.

SEQ ID NO: 21 is a 24374 cry1Ab expression cassette.

SEQ ID NO: 22 is a 24375 cry1Ab expression cassette.

SEQ ID NO: 23 is a 24376 cry1Ab expression cassette.

SEQ ID NO: 24 is a 24377 cry1Ab expression cassette.

SEQ ID NO: 25 is a 24379 cry1Ab expression cassette.

SEQ ID NO: 26 is a 24380 cry1Ab expression cassette.

SEQ ID NO: 27 is a 24381 cry1Ab expression cassette.

SEQ ID NO: 28 is a 24383 cry1Ab expression cassette.

SEQ ID NO: 29 is a motif miR159h3p initiator RNA sequence.

SEQ ID NO: 30 is a tgene miR159h-3p initiator RNA sequence.

SEQ ID NO: 31 is a motif miR156i-3p initiator RNA sequence.

SEQ ID NO: 32 is a motif miR171i-5p initiator RNA sequence.

SEQ ID NO: 33 is a motif miR396b-3p initiator RNA sequence.

SEQ ID NO: 34 is a tgene miR396b-3p initiator RNA sequence.

SEQ ID NO: 35 is a mutant miR159h-3p RNA sequence.

SEQ ID NO: 36 is a miR159h-3p RNA sequence.

SEQ ID NO: 37 is a miR156i-3p RNA sequence.

SEQ ID NO: 38 is a miR171i-5p RNA sequence.

SEQ ID NO: 39 is a miR396b-3p RNA sequence.

SEQ ID NO: 40 is a mutant miR396b-3p RNA sequence.

SEQ ID NO: 41 is a motif miR159h-3p initiator DNA sequence.

SEQ ID NO: 42 is a tgene miR159h-3p initiator DNA sequence.

SEQ ID NO: 43 is a motif miR156i-3p initiator DNA sequence.

SEQ ID NO: 44 is a motif miR171i-5p initiator DNA sequence.

SEQ ID NO: 45 is a motif miR396b-3p initiator DNA sequence.

SEQ ID NO: 46 is a tgene miR396b-3p initiator DNA sequence.

SEQ ID NO: 47 is a vector 24366 nucleotide sequence.

SEQ ID NO: 48 is a vector 24373 nucleotide sequence.

SEQ ID NO: 49 is a vector 24374 nucleotide sequence.

SEQ ID NO: 50 is a vector 24375 nucleotide sequence.

SEQ ID NO: 51 is a vector 24376 nucleotide sequence.

SEQ ID NO: 52 is a vector 24377 nucleotide sequence.

SEQ ID NO: 53 is a vector 24379 nucleotide sequence.

SEQ ID NO: 54 is a vector 24380 nucleotide sequence.

SEQ ID NO: 55 is a vector 24381 nucleotide sequence.

SEQ ID NO: 56 is a vector 24383 nucleotide sequence.

SEQ ID NO: 57 is a vector 24372 nucleotide sequence.

SEQ ID NO: 58 is a vector 24378 nucleotide sequence.

SEQ ID NO: 59 is a vector 24382 nucleotide sequence.

SEQ ID NO: 60 is a mCry1Ab-17 amino acid sequence.

SEQ ID NO: 61 is a 24373 mCry1Ab-17 amino acid sequence.

SEQ ID NO: 62 is a 24374 mCry1Ab-17 amino acid sequence.

SEQ ID NO: 63 is a 24375 mCry1Ab-17 amino acid sequence.

SEQ ID NO: 64 is a 24376 mCry1Ab-17 amino acid sequence.

SEQ ID NO: 65 is a 24377 mCry1Ab-17 amino acid sequence.

SEQ ID NO: 66 is a 24379 mCry1Ab-17 amino acid sequence.

SEQ ID NO: 67 is a mCry1Ab-17 nucleotide sequence.

SEQ ID NO: 68 is a 24373 mCry1Ab-17 nucleotide sequence.

SEQ ID NO: 69 is a 24374 mCry1Ab-17 nucleotide sequence.

SEQ ID NO: 70 is a 24375 mCry1Ab-17 nucleotide sequence.

SEQ ID NO: 71 is a 24376 mCry1Ab-17 nucleotide sequence.

SEQ ID NO: 72 is a 24377 mCry1Ab-17 nucleotide sequence.

SEQ ID NO: 73 is a 24379 mCry1Ab-17 nucleotide sequence.

SEQ ID NO: 74 is an RNA sequence of a 23708miR2275a-3p target insertion sequence.

SEQ ID NO: 75 is an RNA sequence of a 23711miR2275b-5p target insertion sequence.

SEQ ID NO: 76 is an RNA sequence of a miR2275b-3p target insertion sequence in vector 23712, 23713 and 23714.

SEQ ID NO: 77 is an RNA sequence of a 23715miR2275b-3p target insertion sequence.

SEQ ID NO: 78 is an RNA sequence of a 23716miR2275b-3p target insertion sequence.

SEQ ID NO: 79 is a DNA sequence of a 23708miR2275a-3p target insertion sequence.

SEQ ID NO: 80 is a DNA sequence of a 23711miR2275b-5p target insertion sequence.

SEQ ID NO: 81 is a DNA sequence of a miR2275b-3p target insertion sequence in vector 23712, 23713 and 23714.

SEQ ID NO: 82 is a DNA sequence of a 23715miR2275b-3p target insertion sequence.

SEQ ID NO: 83 is a DNA sequence of a 23716miR2275b-3p target insertion sequence.

SEQ ID NO: 84 is a 23705 vip3 expression cassette nucleotide sequence.

SEQ ID NO: 85 is a 23708 vip3 expression cassette nucleotide sequence.

SEQ ID NO: 86 is a 23711 vip3 expression cassette nucleotide sequence.

SEQ ID NO: 87 is a 23712 vip3 expression cassette nucleotide sequence.

SEQ ID NO: 88 is a 23713 vip3 expression cassette nucleotide sequence.

SEQ ID NO: 89 is a 23714 vip3 expression cassette nucleotide sequence.

SEQ ID NO: 90 is a 23715 vip3 expression cassette nucleotide sequence.

SEQ ID NO: 91 is a 23716 vip3 expression cassette nucleotide sequence.

SEQ ID NO: 92 is a 23717 vip3 expression cassette nucleotide sequence.

SEQ ID NO: 93 is a 23718 vip3 expression cassette nucleotide sequence.

SEQ ID NO: 94 is a miR2275a-3p initiator RNA sequence.

SEQ ID NO: 95 is a miR2275b-5p initiator RNA sequence.

SEQ ID NO: 96 is a miR2275b-3p initiator RNA sequence.

SEQ ID NO: 97 is a miR2275b-3p-target initiator RNA sequence.

SEQ ID NO: 98 is a miR2275a-3p initiator DNA sequence.

SEQ ID NO: 99 is a miR2275b-5p initiator DNA sequence.

SEQ ID NO: 100 is a miR2275b-3p initiator DNA sequence.

SEQ ID NO: 101 is a miR2275b-3p-target initiator DNA sequence.

SEQ ID NO: 102 is a miRNA2275b-3p RNA sequence.

SEQ ID NO: 103 is a miR2275b-5p RNA sequence.

SEQ ID NO: 104 is vector 23705 nucleotide sequence.

SEQ ID NO: 105 is vector 23708 nucleotide sequence.

SEQ ID NO: 106 is vector 23711 nucleotide sequence.

SEQ ID NO: 107 is vector 23712 nucleotide sequence.

SEQ ID NO: 108 is vector 23713 nucleotide sequence.

SEQ ID NO: 109 is vector 23714 nucleotide sequence.

SEQ ID NO: 110 is vector 23715 nucleotide sequence.

SEQ ID NO: 111 is vector 23716 nucleotide sequence.

SEQ ID NO: 112 is vector 23717 nucleotide sequence.

SEQ ID NO: 113 is vector 23718 nucleotide sequence.

SEQ ID NO: 114 is a 23705 Vip3 amino acid sequence.

SEQ ID NO: 115 is a 23716 Vip3 amino acid sequence.

SEQ ID NO: 116 is a 23717 Vip3 amino acid sequence.

SEQ ID NO: 117 is a 23718 Vip3 amino acid sequence.

SEQ ID NO: 118 is a 23705 Vip3 nucleotide sequence.

SEQ ID NO: 119 is a 23716 Vip3 nucleotide sequence.

SEQ ID NO: 120 is a 23717 Vip3 nucleotide sequence.

SEQ ID NO: 121 is a 23718 Vip3 nucleotide sequence.

SEQ ID NO: 122 is tgene input sequence.

SEQ ID NO: 123 is a tgene output sequence.

SEQ ID NO: 124 is a motif input sequence.

SEQ ID NO: 125 is a motif output sequence.

### DETAILED DESCRIPTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; l), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

For clarity, certain terms used in the specification are defined and presented as follows:

As used herein and in the appended claims, the singular forms “a,” “an,” and “the” include plural reference unless the context clearly dictates otherwise. Thus, for example, reference to “a plant” is a reference to one or more plants and includes equivalents thereof known to those skilled in the art, and so forth.

As used herein, the term “and/or” refers to and encompasses any and all possible combinations of one or more of the associated listed items, as well as the lack of combinations when interpreted in the alternative, “or.”

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1° C., preferably ±0.5° C. Where the term “about” is used in the context of this invention (e.g., in combinations with temperature or molecular weight values) the exact value (i.e., without “about”) is preferred.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

“Activity” of the insecticidal proteins of the invention is meant that the insecticidal proteins function as orally active insect control agents, have a toxic effect, and/or are able to disrupt or deter insect feeding, which may or may not cause death of the insect. When an insecticidal protein of the invention is delivered to the insect, the result is typically death of the insect, or the insect does not feed upon the source that makes the insecticidal protein available to the insect. “Pesticidal” is defined as a toxic biological activity capable of controlling a pest, such as an insect, nematode, fungus, bacteria, or virus, preferably by killing or destroying them. “Insecticidal” is defined as a toxic biological activity capable of controlling insects, preferably by killing them. A “pesticidal agent” is an agent that has pesticidal activity. An “insecticidal agent” is an agent that has insecticidal activity.

As used herein, the terms “backcross” and “backcrossing” refer to the process whereby a progeny plant is crossed back to one of its parents for one or more generations (e.g., 2 or more, 3 or more, 4 or more, 5 or more, 6 or more, or 7 or more times, etc.). In a backcrossing scheme, the “donor” parent refers to the parental plant with the desired gene or DNA construct or locus to be introgressed. The “recipient” parent (used one or more times) or “recurrent” parent (used two or more times) refers to the parental plant into which the gene or DNA construct or locus is being introgressed. For example, see Ragot et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on. In embodiments, at least one or more generations of progeny are identified and/or selected for the presence of the desired gene or locus (e.g., in a nucleic acid sample from the progeny plant or plant part). In embodiments, two or more generations (or even all generations) of progeny are identified and/or selected for the presence of the desired gene or DNA construct or locus.

The term “chimeric construct” or “chimeric gene” or “chimeric polynucleotide” or “chimeric nucleic acid” or “chimeric protein” (or similar terms) as used herein refers to a construct or nucleic acid molecule or protein comprising two or more polynucleotides or amino acid motifs or domains, respectively, of different origin assembled into a single nucleic acid molecule or protein. The term “chimeric construct”, “chimeric gene”, “chimeric polynucleotide” or “chimeric nucleic acid” refers to any construct or molecule that contains, without limitation, (1) polynucleotides (e.g., DNA), including regulatory and coding polynucleotides that are not found together in nature (i.e., at least one of the polynucleotides in the construct is heterologous with respect to at least one of its other polynucleotides), or (2) polynucleotides encoding parts of proteins not naturally adjoined, or (3) parts of promoters that are not naturally adjoined. Further, a chimeric construct, chimeric gene, chimeric polynucleotide or chimeric nucleic acid may comprise regulatory polynucleotides and coding polynucleotides that are derived from different sources, or comprise regulatory polynucleotides and coding polynucleotides derived from the same source, but arranged in a manner different from that found in nature. In some embodiments of the invention, the chimeric construct, chimeric gene, chimeric polynucleotide or chimeric nucleic acid comprises an expression cassette comprising a polynucleotide of the invention under the control of regulatory polynucleotides, particularly under the control of regulatory polynucleotides functional in plants or bacteria.

A “coding sequence” is a nucleic acid sequence that is transcribed into RNA such as mRNA, rRNA, tRNA, snRNA, sense RNA or antisense RNA. In some aspects, the RNA is then translated in an organism, such as a corn plant, to produce a protein, e.g. an insecticidal protein of the invention. In other aspects, the RNA is not translated to produce a protein but functions as an RNA molecule to modulate expression of a recombinant insecticidal protein of the invention.

As used herein, the term “completely fertile” refers to a plant that is at least as fertile as a control plant (e.g., one or both of its parents, a near isogenic plant that lacks a vip3 coding sequence of the invention, and so forth). In some embodiments, “completely fertile” plants release at least as many pollen grains per tassel per day in the three-day period immediately following anther extrusion as the control plant. In some embodiments, “completely fertile” plants release more pollen grains per tassel per day in the three-day period immediately following anther extrusion than the control plant.

As used herein, a “codon optimized” sequence means a nucleotide sequence wherein the codons are chosen to reflect the particular codon bias that a host cell or organism may have. This is typically done in such a way so as to preserve the amino acid sequence of the polypeptide encoded by the nucleotide sequence to be optimized. In certain embodiments, a DNA sequence of a recombinant DNA construct of the invention includes codons optimized for a cell (e.g., an animal, plant, or fungal cell) in which the construct is to be expressed. For example, a construct to be expressed in a plant cell can have all or parts of its sequence (e.g., the first gene suppression element or the gene expression element) codon optimized for expression in a plant. See, for example, U.S. Pat. No. 6,121,014, incorporated herein by reference.

The terms “comprises” or “comprising,” when used in this specification, specify the presence of stated features, integers, steps, operations, elements, or components, but do not preclude the presence or addition of one or more other features, integers, steps, operations, elements, components, or groups thereof.

As used herein, the transitional phrase “consisting essentially of (and grammatical variants) means that the scope of a claim is to be interpreted to encompass the specified materials or steps recited in the claim” and those that do not materially alter the basic and novel characteristic(s)” of the claimed invention. Thus, the term “consisting essentially of” when used in a claim of this invention is not intended to be interpreted to be equivalent to “comprising.”

To “control” insects means to inhibit, through a toxic effect, the ability of insect pests to survive, grow, feed, or reproduce, or to limit insect-related damage or loss in crop plants or to protect the yield potential of a crop when grown in the presence of insect pests. To “control” insects may or may not mean killing the insects, although it preferably means killing the insects.

As used herein, the term “corn” is synonymous with the term “maize” or “Zea mays.”

In the context of the invention, “corresponding to” or “corresponds to” means that when the amino acid sequences of variant or homolog Cry proteins are aligned with each other, the amino acids that “correspond to” certain enumerated positions in the variant or homolog protein are those that align with these positions in a reference protein but that are not necessarily in these exact numerical positions relative to the particular reference amino acid sequence of the invention. For example, if SEQ ID NO: 114 is the reference sequence and is aligned with SEQ ID NO: 115, amino acid Phe at position 201 (Phe201) of SEQ ID NO: 115 “corresponds to” a Phe at position 183 (Phe183) of SEQ ID NO: 114, or for example, Glu199 of SEQ ID NO: 117 “corresponds to” Lys195 of SEQ ID NO: 114.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide group.

“Effective insect-controlling amount” means that concentration of an insecticidal protein that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects.

“Expression cassette” as used herein means a nucleic acid sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may have at least one of its components heterologous with respect to at least one of its other components. The expression cassette may also be one that is naturally occurring but has been obtained in a recombinant form useful for heterologous expression. Such usage of an expression cassette makes it so it is not naturally occurring in the cell into which it has been introduced. Typically, however, the expression cassette is heterologous with respect to the host, i.e., the particular nucleic acid sequence of the expression cassette does not occur naturally in the host cell and must have been introduced into the host cell or an ancestor of the host cell by a transformation process. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used. Any available terminator known to function in plants can be used in the context of the invention.

The term “expression” when used with reference to a polynucleotide, such as a gene, open reading frame (ORF) or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In some embodiments of the invention, “expression” refers to the transcription and stable accumulation of sense (mRNA) or functional RNA. In some embodiments of the invention, “expression” refers to the production of protein.

A “gene” is a defined region that is located within a genome and comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

“Gene of interest” refers to any nucleic acid molecule which, when transferred to a plant, confers upon the plant a desired trait such as antibiotic resistance, virus resistance, insect resistance, disease resistance, or resistance to other pests, herbicide tolerance, abiotic stress tolerance, male sterility, modified fatty acid metabolism, modified carbohydrate metabolism, improved nutritional value, improved performance in an industrial process or altered reproductive capability. The “gene of interest” may also be one that is transferred to plants for the production of commercially valuable enzymes or metabolites in the plant.

A “heterologous” nucleic acid sequence or nucleic acid molecule is a nucleic acid sequence or nucleic acid molecule not naturally associated with a host cell into which it is introduced, including non-naturally occurring multiple copies of a naturally occurring nucleic acid sequence. A heterologous nucleic acid sequence or nucleic acid molecule may comprise a chimeric sequence such as a chimeric expression cassette, where the promoter and the coding region are derived from multiple source organisms. The promoter sequence may be a constitutive promoter sequence, a tissue-specific promoter sequence, a chemically-inducible promoter sequence, a wound-inducible promoter sequence, a stress-inducible promoter sequence, or a developmental stage-specific promoter sequence.

A “homologous” nucleic acid sequence is a nucleic acid sequence naturally associated with a host cell into which it is introduced.

The term “identity” or “identical” or “substantially identical,” in the context of two nucleic acid or amino acid sequences, refers to two or more sequences or subsequences that have at least 60%, preferably at least 80%, more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues or bases in length, more preferably over a region of at least about 100 residues or bases, and most preferably the sequences are substantially identical over at least about 150 residues or bases. In an especially preferred embodiment, the sequences are substantially identical over the entire length of the coding regions. Furthermore, substantially identical nucleic acid or amino acid sequences perform substantially the same function.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (National Center for Biotechnology Information, U.S. National Library of Medicine, 8600 Rockville Pike, Bethesda, Md. 20894 USA). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but not to other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone homologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A further indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions.

The term “isolated” nucleic acid molecule, polynucleotide or protein is a nucleic acid molecule, polynucleotide or protein that no longer exists in its natural environment. An isolated nucleic acid molecule, polynucleotide or protein of the invention may exist in a purified form or may exist in a recombinant host such as in a transgenic bacteria or a transgenic plant. Therefore, a claim to an “isolated” nucleic acid molecule, as enumerated herein, encompasses a nucleic acid molecule that is comprised within a transgenic plant genome.

As used herein, the term “microRNA” (miRNA) refers to small, non-coding RNA gene products of approximately 18-26 nucleotides in length and found in diverse organisms, including animals and plants. miRNAs structurally resemble siRNAs except that they arise from structured, foldback-forming precursor transcripts derived from miRNA genes. Primary transcripts of miRNA genes form hairpin structures that are processed by the multidomain RNaseIII-like nuclease DICER and DROSHA (in animals) or DICER-LIKE1 (DCL1; in plants) to yield miRNA duplexes. The mature miRNA is incorporated into RISC complexes after duplex unwinding Plant miRNAs interact with their RNA targets by first binding to an initiator sequence that has perfect or near perfect complementarity to the miRNA.

A “nucleic acid molecule” or “nucleic acid sequence” is a segment of single- or double-stranded DNA or RNA that can be isolated from any source. In the context of the invention, the nucleic acid molecule is typically a segment of DNA. In some embodiments, the nucleic acid molecules of the invention are isolated nucleic acid molecules.

“Operably linked” refers to the association of polynucleotides on a single nucleic acid fragment so that the function of one affects the function of the other. For example, a promoter is operably linked with a coding polynucleotide or functional RNA when it is capable of affecting the expression of that coding polynucleotide or functional RNA (i.e., that the coding polynucleotide or functional RNA is under the transcriptional control of the promoter). Coding polynucleotides in sense or antisense orientation can be operably linked to regulatory polynucleotides.

As used herein “pesticidal,” insecticidal,” and the like, refer to the ability of a Cry protein or a vegetative insecticidal protein (Vip) of the invention to control a pest organism or an amount of a Cry protein or Vip that can control a pest organism as defined herein. Thus, a pesticidal Cry protein or Vip can kill or inhibit the ability of a pest organism (e.g., insect pest) to survive, grow, feed, or reproduce.

The terms “protein,” “peptide” and “polypeptide” are used interchangeably herein.

A “plant” is any plant at any stage of development, particularly a seed plant.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue. For example, the “tapetum” is a tissue within the sporangium, especially the anther, of corn plants that provides nutrition for growing spores.

A “polynucleotide” refers to a polymer composed of many nucleotide monomers covalently bonded in a chain. Such “polynucleotides” includes DNA, RNA, modified oligo nucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

“Polynucleotide of interest” refers to any polynucleotide which, when transferred to an organism, e.g., a plant, confers upon the organism a desired characteristic such as insect resistance, disease resistance, herbicide tolerance, antibiotic resistance, improved nutritional value, improved performance in an industrial process, production of commercially valuable enzymes or metabolites or altered reproductive capability.

A “promoter” is an untranslated DNA sequence upstream of the coding region that contains the binding site for RNA polymerase and initiates transcription of the DNA. The promoter region may also include other elements that act as regulators of gene expression.

As used herein, the term “recombinant” refers to a form of nucleic acid (e.g., DNA or RNA) or protein or an organism that would not normally be found in nature and as such was created by human intervention. As used herein, a “recombinant nucleic acid molecule” is a nucleic acid molecule comprising a combination of polynucleotides that would not naturally occur together and is the result of human intervention, e.g., a nucleic acid molecule that is comprised of a combination of at least two polynucleotides heterologous to each other, or a nucleic acid molecule that is artificially synthesized, for example, a polynucleotide synthesize using an assembled nucleotide sequence, and comprises a polynucleotide that deviates from the polynucleotide that would normally exist in nature, or a nucleic acid molecule that comprises a transgene artificially incorporated into a host cell's genomic DNA and the associated flanking DNA of the host cell's genome. Another example of a recombinant nucleic acid molecule is a DNA molecule resulting from the insertion of a transgene into a plant's genomic DNA, which may ultimately result in the expression of a recombinant RNA or protein molecule in that organism. As used herein, a “recombinant plant” is a plant that would not normally exist in nature, is the result of human intervention, and contains a transgene or heterologous nucleic acid molecule incorporated into its genome. As a result of such genomic alteration, the recombinant plant is distinctly different from the related wild-type plant.

“Regulatory elements” refer to sequences involved in controlling the expression of a nucleotide sequence. Regulatory elements comprise a promoter operably linked to the nucleotide sequence of interest and termination signals. They also typically encompass sequences required for proper translation of the nucleotide sequence.

“Transformation” is a process for introducing heterologous nucleic acid into a host cell or organism. In particular embodiments, “transformation” means the stable integration of a DNA molecule into the genome (nuclear or plastid) of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

The invention relates generally to methods of modulating, for example reducing, recombinant insecticidal protein expression in male reproductive cells and/or tissues of transgenic plants, recombinant DNA constructs useful in such methods, as well as transgenic plants, plant parts, cells, and seeds containing such recombinant DNA constructs. The recombinant DNA constructs and the transgenic plants, plant parts, cells, and seeds containing such constructs provide a greatly improved way to minimize any potential risks to non-pest insect species associated with expression of insecticidal proteins in pollen or for mitigating the impact that expression of insecticidal proteins in pollen and/or tapetum may have on male fertility in transgenic plants.

In some embodiments, the invention provides a recombinant DNA construct that comprises an insecticidal protein-coding sequence operably linked to a DNA sequence encoding a microRNA (miRNA) element that is capable of binding to a plant microRNA that is male tissue-specific or male tissue-preferred in a corn plant, i.e. a chimeric transgene including an insecticidal protein-coding sequence encoding the recombinant insecticidal protein and at least one miRNA element comprising at least one male tissue-specific or male tissue-preferred miRNA initiator sequence operably linked to the protein-coding sequence. In other embodiments, the recombinant DNA construct is useful for suppressing the expression of a recombinant insecticidal protein in a male reproductive tissue of a transgenic plant, such as a transgenic corn plant. In other embodiments, the invention provides a recombinant DNA molecule comprising the recombinant DNA construct and methods of use thereof. Nucleic acid sequences of the invention can be provided as DNA or as RNA, as specified; disclosure of one necessarily defines the other, as is known to one of ordinary skill in the art. Furthermore, disclosure of a given nucleic acid sequence necessarily defines the exact complement of that sequence, as is known to one of ordinary skill in the art.

A “male tissue-specific or “male tissue-preferred” miRNA initiator sequence is a small RNA of about 18 to about 26 nucleotides (e.g., 18, 19, 20, 21, 22, 23, 24, 25 or 26 nucleotides) that is recognized and bound by an endogenous plant microRNA that is enriched (male tissue-preferred) or specifically expressed (male tissue-specific) in one or more male reproductive tissue(s) (e.g., pollen and/or tapetum) of a plant, i.e., having a male tissue-specific or male tissue-preferred expression pattern. Male tissue-specific or male tissue-preferred miRNAs are naturally occurring in plants and can be detected using techniques known in the art, such as low molecular weight northern analysis. Examples of miRNA initiator sequences for endogenous corn plant microRNAs are provided as SEQ ID NO: 29-34 and SEQ ID NO: 94-97. Examples of DNA sequences that encode such miRN initiator sequences are provided as SEQ ID NO: 41-46 and SEQ ID NO: 98-101, respectively. In some embodiments, a miRNA initiator sequence is the exact DNA complement (with no mismatches) to a given male tissue-specific or male tissue-preferred microRNA. Such miRNA initiator sequences are designated herein as “motif” sequences. In other embodiments, a miRNA initiator sequence varies by at least 1-3 nucleotide mismatches compared to a given male tissue-specific or male tissue-preferred microRNA, which are designated herein as “tgene” sequences because the design of the initiator sequence is based on the predicted sequence of an endogenous target gene. These “tgene” miRNA initiator sequences nonetheless have sufficient complementarity to bind or hybridize, e.g., under typical physiological conditions, to the intended microRNA. “Complementarity” refers to the capability of nucleotides on one polynucleotide strand to base-pair with nucleotides on another polynucleotide strand according to the standard Watson-Crick complementarity rules (i.e., guanine pairs with cytosine (G:C) and adenine pairs with either thymine (A:T) or uracil (A:U); it is possible for intra-strand hybridization to occur between two or more complementary regions of a single polynucleotide. When included in a recombinant DNA construct as described herein, a miRNA initiator sequence is capable of RNAi-mediated suppression or disruption of the expression of a transgene RNA or the expression of a recombinant protein, such as a recombinant insecticidal protein.

In some embodiments of the invention, a microRNA of the invention and thus a miRNA initiator sequence of the invention is selectively or preferentially functional in pollen tissue or tapetum tissue.

In some embodiments of the invention, a DNA construct of the invention comprises a miRNA element within a 5′ untranslated region of an insecticidal protein-coding sequence. In other embodiments, a DNA construct of the invention comprises a miRNA element within a 3′ untranslated region of an insecticidal protein-coding sequence. In still other embodiments, a DNA construct of the invention comprises a miRNA element within an insecticidal protein-coding sequence between the start and stop codons. In other embodiments, the miRNA element is located between the protein-coding sequence and a polyadenylation sequence which is part of a 3′ untranslated region.

In some embodiments of the invention, a DNA construct of the invention comprises at least one, at least two, at least three, or more than three miRNA elements. Each of the miRNA elements may comprise at least one, at least two, at least three, or more than three miRNA initiator sequence(s). A miRNA element may be any length but preferably they are from about 40 nucleotides to about 80 nucleotides. In other embodiments, a miRNA element of the invention comprises a sequence that encodes a miRNA initiator sequence selected from the group consisting of SEQ ID NO: 29-34 or SEQ ID NO: 94-97. In other embodiments, a sequence that encodes a miRNA initiator sequence of the invention is selected from the group consisting of SEQ ID NO: 41-46 or SEQ ID NO: 98-101. In still other embodiments, the miRNA element comprises a target insertion sequence comprising a miRNA initiator sequence and a synthetic nucleotide sequence flanking the 5′ and/or the 3′ end of the initiator sequence. Such flanking sequences may be useful for allowing a microRNA to have better access to the miRNA initiator sequence. On the 5′ end of the initiator sequence, flank size can be about 40, or about 30, or about 20, 19, 18, 17, 16 or 15 nucleotides, but preferably 17 nucleotides. On the 3′ end of the initiator sequence flank size can range from about 40, or about 30, or about 20, 19, 18, 17, 16, 15, 13, 12 or 11 nucleotides, but preferably 13 nucleotides. In other embodiments, a sequence that comprises an initiator sequence of the invention and synthetic flanking sequences on the 5′ and/or 3′ end of the initiator sequence is a target insertion sequence of the invention. In other embodiments, a target insertion sequence is selected from the group consisting of SEQ ID NO: 10-18 or SEQ ID NO: 79-83. Thus, a miRNA element of the invention may comprise only a miRNA initiator sequence or it may comprise a target insertion sequence and any combination thereof.

In some embodiments, the miRNA elements of the invention are synthesized or modified in vitro to contain more, fewer, or different miRNA initiator sequences or flanking sequences and/or to rearrange the relative position of one or more miRNA initiator sequence(s) or flanking sequences, where such a modification is beneficial in increasing or decreasing the effect of the miRNA element. Methods for synthesizing or for in vitro modification of a miRNA element and determining the optimal variation for the desired level of suppression are known by those of skill in the art. Chimeric miRNA elements can also be designed using methods known to those of skill in the art, such as by inserting additional desired miRNA initiator sequences internally in an miRNA element or by linking additional miRNA initiator sequences 5′ or 3′ to an miRNA element.

In some embodiments, expression of a miRNA initiator sequence in a transgenic corn plant reduces the expression of a recombinant insecticidal protein of the invention in male reproductive tissue, such as pollen and/or tapetum, of the transgenic corn plant compared to non-male reproductive tissue in the transgenic corn, such leaf tissue. Reduction of recombinant insecticidal protein expression in a cell or tissue as used herein refers to the decreased or suppressed level of a recombinant insecticidal protein in a cell or tissue as compared to a reference cell or tissue by at least about 25%, at least about 35, at least about 45%, at least about 55%, at least about 65%, at least about 75%, at least about 80%, at least about 85%, at least about 90%, at least about 95%, or at least about 99%. A reference cell or tissue can be, e.g., a vegetative cell or tissue from the same or a similar transgenic plant expressing the recombinant insecticidal protein, or e.g., a vegetative cell or tissue from a transgenic plant having a similar transgene for expressing the recombinant insecticidal protein but lacking the miRNA element. Reduction in insecticidal protein expression can be determined using any technique known to one skilled in the art, such as by directly measuring protein accumulation in a cell or tissue sample using a technique such as ELISA or western blot analysis, by measuring biological activity of the insecticidal protein, or by phenotypically determining insecticidal protein expression. In some embodiments, reduction of recombinant insecticidal protein refers to a sufficient reduction in expression of a recombinant insecticidal protein in a male reproductive tissue of a transgenic corn plant, resulting in a detectable phenotype of increased male fertility in the transgenic corn plant compared to a suitable control corn plant. The detection of increased male fertility in such a transgenic corn plant would therefore indicate the selective reduction of the recombinant insecticidal protein.

In other embodiments, expression of a miRNA initiator sequence of the invention in a transgenic corn plant increases the expression of a recombinant insecticidal protein of the invention in male reproductive tissue, such as pollen or tapetum, of the transgenic corn plant compared to non-male reproductive tissue in the transgenic corn, such leaf tissue. Increased recombinant insecticidal protein expression in a cell or tissue as used herein refers to the increased level of a recombinant insecticidal protein in a cell or tissue as compared to a reference cell or tissue by at least about 25%, at least about 35, at least about 45%, at least about 55%, at least about 65%, at least about 75%, at least about 80%, at least about 85%, at least about 90%, at least about 95%, or at least about 99%. A reference cell or tissue can be, e.g., a vegetative cell or tissue from the same or a similar transgenic plant expressing the recombinant insecticidal protein, or e.g., a vegetative cell or tissue from a transgenic plant having a similar transgene for expressing the recombinant insecticidal protein but lacking the miRNA element. An increase in insecticidal protein expression can be determined using any technique known to one skilled in the art, such as by directly measuring protein accumulation in a cell or tissue sample using a technique such as ELISA or western blot analysis, by measuring biological activity of the insecticidal protein, or by phenotypically determining insecticidal protein expression.

In some embodiments, expression in a transgenic corn plant of a recombinant insecticidal protein encoded by a DNA construct of the invention confers insect pest tolerance to the transgenic corn plant in at least vegetative tissues. Such tolerance results from the insecticidal protein causing a reduction in feeding or growth of the insect pest or causes death of the insect pest. In some embodiments, a DNA construct of the invention encodes a recombinant Cry1 insecticidal protein or a recombinant Vip3 insecticidal protein. In other embodiments, the Cry1 protein is a Cry1A insecticidal protein or the Vip3 protein is a Vip3A insecticidal protein. In still other embodiments, the Cry1A protein is a Cry1Ab insecticidal protein or the Vip3A protein is a Vip3Aa insecticidal protein. In still further embodiments, the Cry1Ab insecticidal protein comprises an amino acid sequence selected from the group consisting of SEQ ID NO: 61-66. In still further embodiments, the Vip3Aa protein comprises an amino acid sequence selected from the group consisting of SEQ ID NO: 115-117.

In some embodiments, a recombinant DNA construct of the invention comprises an expression cassette comprising a heterologous promoter operably linked to a recombinant insecticidal protein coding-sequence operably linked to a DNA sequence encoding a microRNA (miRNA) element of the invention that is male tissue-specific or male tissue-preferred in a corn plant. In one embodiment, the expression cassette comprises a sequence selected from the group consisting SEQ ID NO: 20-28 or SEQ ID NO: 85-93.

In some embodiments, the invention provides a recombinant vector comprising a DNA construct of the invention. In other embodiments, the vector comprises a sequence selected from the group consisting of SEQ ID NO: 49-59 or SEQ ID NO: 105-113.

In some embodiments, the invention provides a transgenic corn (maize; Zea mays) plant comprising a DNA construct and/or an expression cassette and/or a recombinant vector of the invention. In other embodiments, the invention provides transgenic seed, progeny or a plant part of the transgenic corn plant of the invention, wherein the transgenic seed, progeny or plant part comprises a DNA construct, expression cassette or recombinant vector of the invention.

In some embodiments, the invention provides a method of making a DNA construct comprising identifying an endogenous male tissue-specific or male tissue-preferred corn microRNA (miRNA); constructing a miRNA element that encodes at least one miRNA initiator sequence that is recognized by said corn miRNA and operably linking said miRNA element to an insecticidal protein-coding sequence. In other embodiments, the male tissue-specific microRNA or male tissue-preferred microRNA is pollen-specific or pollen-preferred or tapetum-specific or tapetum preferred. In other embodiments, the male tissue-specific miRNA element or male tissue-preferred miRNA element is pollen-specific or pollen-preferred or tapetum-specific or tapetum preferred. In other embodiments, the miRNA element comprises a target insertion sequence. In still other embodiments, the target insertion sequence comprises a sequence selected from the group consisting of SEQ ID NO: 10-18 or SEQ ID NO: 79-83. In still further embodiments, the miRNA initiator sequence encoded by said miRNA element comprises a sequence selected from the group consisting of SEQ ID NO: 29-34 or SEQ ID NO: 94-97.

In some embodiments, the invention provides a method of reducing the expression of a recombinant insecticidal protein in a male reproductive tissue of a transgenic corn plant comprising expressing in the transgenic corn plant a DNA construct comprising an insecticidal protein-coding sequence encoding the recombinant insecticidal protein and at least a first male tissue-specific miRNA element or a male tissue-preferred miRNA element operably linked to the insecticidal protein-coding sequence, wherein the miRNA element encodes at least one miRNA initiator sequence. In other embodiments, the miRNA element is comprised within a 5′ untranslated region of the insecticidal protein-coding sequence, or within a 3′ untranslated region of the insecticidal protein-coding sequence or within the insecticidal protein-coding sequence between the start and stop codons. In other embodiments, the miRNA element comprises at least two, at least three, or more than three miRNA initiator sequences. In still other embodiments, the male tissue is pollen and/or tapetum. In still other embodiments, the miRNA initiator sequence is selected from the group consisting of SEQ ID NO: 29-34 or SEQ ID NO: 94-97. In still other embodiments, the miRNA element comprises a target insertion sequence that is selected from the group consisting of SEQ ID NO: 10-18 or SEQ ID NO: 79-83.

In some embodiments of a method to selectively reduce the expression of a recombinant insecticidal protein in male reproductive tissue of a transgenic corn plant, the expression of the insecticidal protein in the transgenic corn plant confers insect pest tolerance to the corn plant in at least the vegetative tissue, such as leaf tissue, of the plant. In other embodiments, the insecticidal protein-coding sequence encodes a Cry1 protein or a Vip3 protein. In other embodiments, the insecticidal protein-coding sequence encodes a Cry1A protein or a Vip3A protein. In still other embodiments, the insecticidal protein-coding sequence encodes a Cry1Ab protein or a Vip3Aa protein. In still other embodiments, the insecticidal protein-coding sequence encodes a Cry1Ab protein comprising an amino acid sequence selected from the group consisting of SEQ ID NO: 61-66. In other embodiments, the insecticidal protein-coding sequence encodes a Vip3Aa protein comprising an amino acid sequence selected from the group consisting of SEQ ID NO: 115-117. In still other embodiments, the DNA construct comprises an expression cassette comprising a sequence selected from the group consisting of SEQ ID NO: 20-28 or SEQ ID NO: 85-93.

In some embodiments, the invention provides a method of producing a Vip3-expressing corn plant having increased male fertility, comprising introducing into the genome of a corn plant part a recombinant DNA construct comprising a Vip3 protein-coding sequence operably linked to a DNA sequence encoding a microRNA (miRNA) element that is male tissue-specific or male tissue-preferred in a corn plant, such tapetum-specific or tapetum-preferred, wherein the miRNA element is heterologous with respect to the Vip3 protein-coding sequence; producing a corn plant from the corn plant part, wherein the miRNA element reduces the expression of the Vip3 protein in the male reproductive tissue, e.g. tapetum, thereby producing a Vip3-expressing corn plant having increased male fertility compared to a Vip3-expressing corn plant without the DNA construct.

In some embodiments, the invention provides a method of producing a Vip3-expressing corn plant having increased male fertility, comprising: (a) crossing a first Vip3-expressing corn plant with a second corn plant, wherein the first corn plant comprises within its genome a recombinant DNA construct comprising a Vip3 protein-coding sequence operably linked to a DNA sequence encoding a microRNA (miRNA) element that is male tissue-specific or male tissue-preferred in a corn plant, wherein the miRNA element is heterologous with respect to the Vip3 protein-coding sequence; and optionally (b) backcrossing a resulting Vip3-epressing progeny corn plant of step (a) comprising the DNA construct with a parent plant to produce backcross progeny plants; (c) selecting for backcross a Vip3-expressing progeny plant that comprises the DNA construct; and (d) performing steps b) and c) at least three times to fix the DNA construct in a desired genetic background, thereby producing a Vip3-expressing corn plant comprising the DNA construct and having increased male fertility compared to a Vip3-expressing maize plant without the DNA construct.

In some embodiments, the invention provides a method of improving seed production from a Vip3-expressing corn plant, comprising: (a) crossing a first corn plant or corn germplasm with a second corn plant or corn germplasm, wherein the first or second corn plant or corn germplasm expresses a Vip3 insecticidal protein, and wherein the first corn plant or corn germplasm comprises within its genome a recombinant DNA construct comprising a Vip3 protein-coding sequence operably linked to a DNA sequence encoding a microRNA (miRNA) element that is male tissue-specific or male tissue-preferred in a corn plant, wherein the miRNA element is heterologous with respect to the Vip3 protein-coding sequence, and wherein the miRNA element reduces the expression level of the Vip3 protein in the male reproductive tissue; and (b) using a progeny maize plant comprising the DNA construct as a pollinator in a cross with itself or a second corn plant or corn germplasm that functions as a seed parent, thereby improving seed production from the cross as compared with a suitable control cross.

In some embodiments of the above described methods for producing a Vip3-expressing corn plant and improving seed production of a Vip3-expressing corn plant, the miRNA element is comprised within a 5′ untranslated region of said insecticidal protein-coding sequence, or within a 3′ untranslated region of the insecticidal protein-coding sequence or within the insecticidal protein-coding sequence between the start and stop codons. In other embodiments, the male tissue is pollen and/or tapetum. In other embodiments, the miRNA element comprises a nucleotide sequence that encodes at least one miRNA initiator sequence. In still other embodiments the miRNA initiator sequence encoded by the nucleotide sequence is selected from the group consisting of SEQ ID NO: 94-97. In still other embodiments of the methods, the expression of the miRNA initiator sequence in a transgenic corn plant reduces the expression of the Vip3 protein in male reproductive tissue of the transgenic corn plant compared to a non-male reproductive tissue in the transgenic corn plant. In further embodiments, the miRNA element comprises a sequence selected from the group consisting of SEQ ID NO: 74-78.

In some embodiments of the above described methods of the invention, the expression of the Vip3 protein-coding sequence in a transgenic corn plant confers insect pest tolerance to the corn plant. In other embodiments, the insect pest is a fall armyworm or a corn earworm. In other embodiments, the Vip3 protein is a Vip3A protein. In still other embodiments, the Vip3A protein is a Vip3Aa protein. In still other embodiments, the Vip3Aa protein comprises an amino acid sequence selected from the group consisting of SEQ ID NO: 115-117. In still other embodiments of the methods, the DNA construct comprises an expression cassette, wherein the expression cassette comprises a sequence selected from the group consisting of SEQ ID NO: 85-93.

In some embodiments, the invention provides a recombinant insecticidal protein that is active against a lepidopteran insect, wherein the insecticidal protein comprises an amino acid sequence that is encoded by a male tissue-specific or male tissue-preferred miRNA element, and wherein the miRNA element encodes at least one miRNA initiator sequence. In other embodiments, the insecticidal protein is a Cry1 protein or a Vip3 protein. In other embodiments, the Cry1 protein is a Cry1A protein or the Vip3 protein is a Vip3A protein. In still other embodiments, the Cry1A protein is a Cry1Ab protein and the Vip3A protein is a Vip3Aa protein. In still other embodiments, the amino acid sequence is selected from the group consisting of SEQ ID NO: 61-66 or SEQ ID NO: 115-117. In other embodiments, the recombinant insecticidal protein is active against a lepidopteran pest that is a European corn borer, a fall armyworm or a corn earworm. In other embodiments, the male tissue is pollen or tapetum. In other embodiments, the miRNA element comprises a target insertion sequence. In still other embodiments, the miRNA element comprises a target insertion sequence selected from the group consisting of SEQ ID NO: 10-18 or SEQ ID NO: 74-78. In still other embodiments, the miRNA initiator sequence is selected from the group consisting of SEQ ID NO: 29-34 or SEQ ID NO: 94-97.

In some embodiments, the invention provides a synthetic polynucleotide encoding a recombinant insecticidal protein of the invention. In other embodiments, the synthetic polynucleotide comprises a sequence selected from the group consisting of SEQ ID NO: 68-73 or SEQ ID NO: 118-121.

In some embodiments, a DNA construct of the invention is expressed in transgenic plants, thus causing the biosynthesis of the corresponding recombinant insecticidal protein in the transgenic plants. In this way, transgenic plants with enhanced resistance to insects, for example lepidopteran insect pests, are generated. For their expression in transgenic plants, the DNA constructs of the invention may optionally be modified and optimized. Although in many cases genes from microbial organisms can be expressed in plants at high levels without modification, low expression in transgenic plants may result from microbial nucleic acids having codons that are not preferred in plants. It is known in the art that all organisms have specific preferences for codon usage, and the codons of the nucleic acids described in this invention can be changed to conform with plant preferences, while maintaining the amino acids encoded thereby. Furthermore, high expression in plants is best achieved from coding sequences that have at least about 35% GC content, preferably more than about 45%, more preferably more than about 50%, and most preferably more than about 60%. Microbial nucleic acids that have low GC contents may express poorly in plants due to the existence of ATTTA motifs that may destabilize messages, and AATAAA motifs that may cause inappropriate polyadenylation. In some embodiments, sequences can be modified to account for the specific codon preferences and GC content preferences of monocotyledons or dicotyledons as these preferences have been shown to differ (Murray et al. Nucl. Acids Res. 17:477-498 (1989)). In addition, the nucleic acids are screened for the existence of illegitimate splice sites that may cause message truncation. All changes required to be made within the nucleic acids such as those described above can be made using well known techniques of site directed mutagenesis, PCR, and synthetic gene construction, for example, using the methods described in the published patent applications EP 0 385 962, EP 0 359 472, and WO 93/07278.

In some embodiments of the invention, a coding sequence for an insecticidal protein of the invention is made according to the procedure disclosed in U.S. Pat. No. 5,625,136, herein incorporated by reference. In this procedure, maize preferred codons, i.e., the single codon that most frequently encodes that amino acid in maize, are used. The maize preferred codon for a particular amino acid might be derived, for example, from known gene sequences from maize. Maize codon usage for 28 genes from maize plants is found in Murray et al., Nucleic Acids Research 17:477-498 (1989), the disclosure of which is incorporated herein by reference.

In this manner, the nucleotide sequences can be optimized for expression in any plant. It is recognized that all or any part of the gene sequence may be optimized or synthetic. That is, synthetic or partially optimized sequences may also be used.

For more efficient initiation of translation, sequences adjacent to the initiating methionine may be modified. For example, they can be modified by the inclusion of sequences known to be effective in plants. Joshi has suggested an appropriate consensus for plants (NAR 15:6643-6653 (1987)) and Clonetech suggests a further consensus translation initiator (1993/1994 catalog, page 210). These consensus sequences are suitable for use with the nucleic acids of this invention. In embodiments, the sequences are incorporated into constructions comprising the nucleic acids, up to and including the ATG (whilst leaving the second amino acid unmodified), or alternatively up to and including the GTC subsequent to the ATG (with the possibility of modifying the second amino acid of the transgene).

Expression of nucleic acid molecules of the invention in transgenic plants is driven by promoters that function in plants. The choice of promoter will vary depending on the temporal and spatial requirements for expression, and also depending on the target species. Thus, expression of the nucleic acids of this invention in leaves, in stalks or stems, in ears, in inflorescences (e.g. spikes, panicles, cobs, etc.), in roots, and/or seedlings is preferred. In many cases, however, protection against more than one type of insect pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleic acids in the desired cell.

In some embodiments, promoters are used that are expressed constitutively including the actin or ubiquitin or cmp promoters or the CaMV 35S and 19S promoters. The nucleic acids of this invention can also be expressed under the regulation of promoters that are chemically regulated. Preferred technology for chemical induction of gene expression is detailed in the published application EP 0 332 104 (to Ciba-Geigy) and U.S. Pat. No. 5,614,395. A preferred promoter for chemical induction is the tobacco PR-1a promoter.

In other embodiments, a category of promoters which is wound inducible can be used. Numerous promoters have been described which are expressed at wound sites and also at the sites of phytopathogen infection. Ideally, such a promoter should only be active locally at the sites of infection, and in this way the chimeric insecticidal proteins of the invention only accumulate in cells that need to synthesize the proteins to kill the invading insect pest. Preferred promoters of this kind include those described by Stanford et al. Mol. Gen. Genet. 215:200-208 (1989), Xu et al. Plant Molec. Biol. 22:573-588 (1993), Logemann et al. Plant Cell 1:151-158 (1989), Rohrmeier & Lehle, Plant Molec. Biol. 22:783-792 (1993), Firek et al. Plant Molec. Biol. 22:129-142 (1993), and Warner et al. Plant J. 3:191-201 (1993).

Tissue-specific or tissue-preferential promoters useful for the expression of genes encoding chimeric insecticidal proteins of the invention in plants, particularly corn, are those which direct expression in root, pith, leaf or pollen, particularly root. Such promoters, e.g. those isolated from PEPC or trpA, are disclosed in U.S. Pat. No. 5,625,136, or MTL, disclosed in U.S. Pat. No. 5,466,785. Both U. S. patents are herein incorporated by reference in their entirety. In some embodiments, the combination of a male tissue-specific or male tissue-preferred promoter in combination with a DNA construct of the invention, i.e. a DNA construct comprising an insecticidal protein-coding sequence operably linked to a DNA sequence encoding a microRNA (miRNA) element that is male tissue-specific or male tissue-preferred in a corn plant, wherein said miRNA element is heterologous with respect to said insecticidal protein-coding sequence, leads to a larger decrease in expression of the insecticidal protein in pollen or tapetum than a decrease attributable to the tissue-specific/tissue-preferred promoter or miRNA element alone.

In addition, promoters functional in plastids can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of nucleotide sequences of the invention in a plant through the application of an exogenous chemical regulator. Regulation of the expression of nucleotide sequences of the invention via promoters that are chemically regulated enables the polypeptides of the invention to be synthesized only when the crop plants are treated with the inducing chemicals. Depending upon the objective, the promoter may be a chemical-inducible promoter, where application of a chemical induces expression of a nucleotide sequence of the invention, or a chemical-repressible promoter, where application of the chemical represses expression of a nucleotide sequence of the invention.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In further aspects, nucleotide sequences of the invention can be operably associated with a promoter that is wound inducible or inducible by pest or pathogen infection (e.g., a insect or nematode plant pest). Numerous promoters have been described which are expressed at wound sites and/or at the sites of pest attack (e.g., insect/nematode feeding) or phytopathogen infection. Ideally, such a promoter should be active only locally at or adjacent to the sites of attack, and in this way expression of the nucleotide sequences of the invention will be focused in the cells that are being invaded or fed upon. Such promoters include, but are not limited to, those described by Stanford et al., Mol. Gen. Genet. 215:200-208 (1989), Xu et al. Plant Molec. Biol. 22:573-588 (1993), Logemann et al. Plant Cell 1:151-158 (1989), Rohrmeier and Lehle, Plant Molec. Biol. 22:783-792 (1993), Firek et al. Plant Molec. Biol. 22:129-142 (1993), Warner et al. Plant J. 3:191-201 (1993), U.S. Pat. Nos. 5,750,386, 5,955,646, 6,262,344, 6,395,963, 6,703,541, 7,078,589, 7,196,247, 7,223,901, and U.S. Patent Application Publication 2010043102.

In some embodiments of the invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

Numerous other sequences can be incorporated into expression cassettes described in this invention. These include sequences that have been shown to enhance expression such as intron sequences (e.g. from Adhl and bronzel) and viral leader sequences (e.g. from TMV, MCMV and AMV).

It may be preferable to target expression of the nucleic acids of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, the DNA encoding the target peptide from a known organelle-targeted gene product is manipulated and fused upstream of the nucleic acid. Many such target sequences are known for the chloroplast and their functioning in heterologous constructions has been shown. The expression of the nucleic acids of the present invention is also targeted to the endoplasmic reticulum or to the vacuoles of the host cells. Techniques to achieve this are well known in the art.

Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construction of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors comprising the nucleic acid molecules of the present invention may also comprise genes (e.g. phosphomannose isomerase; PMI) which provide for positive selection of the transgenic plants as disclosed in U.S. Pat. Nos. 5,767,378 and 5,994,629, herein incorporated by reference. The choice of selectable marker is not, however, critical to the invention.

In some embodiments, the nucleic acid can be transformed into the nuclear genome. In other embodiments, a nucleic acid of the present invention is directly transformed into the plastid genome. A major advantage of plastid transformation is that plastids are generally capable of expressing bacterial genes without substantial codon optimization, and plastids are capable of expressing multiple open reading frames under control of a single promoter. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Nati. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransferase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleic acid of the present invention is inserted into a plastid-targeting vector and transformed into the plastid genome of a desired plant host. Plants homoplastic for plastid genomes containing a nucleic acid of the present invention are obtained, and are preferentially capable of high expression of the nucleic acid.

In some embodiments, a transgenic plant of the invention may comprise at least a second pesticidal agent which is non-proteinaceous. In some aspects of these embodiments, the second pesticidal agent is an interfering RNA molecule. An interfering RNA typically comprises at least a RNA fragment against a target gene, a spacer sequence, and a second RNA fragment which is complementary to the first, so that a double-stranded RNA structure can be formed. RNA interference (RNAi) occurs when an organism recognizes double-stranded RNA (dsRNA) molecules and hydrolyzes them. The resulting hydrolysis products are small RNA fragments of about 19-24 nucleotides in length, called small interfering RNAs (siRNAs). The siRNAs then diffuse or are carried throughout the organism, including across cellular membranes, where they hybridize to mRNAs (or other RNAs) and cause hydrolysis of the RNA. Interfering RNAs are recognized by the RNA interference silencing complex (RISC) into which an effector strand (or “guide strand”) of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes from which the mRNAs were transcribed. Interfering RNAs are known in the art to be useful for insect control (see, for example, publication WO2013/192256, incorporated by reference herein). An interfering RNA designed for use in insect control produces a non-naturally occurring double-stranded RNA, which takes advantage of the native RNAi pathways in the insect to trigger down-regulation of target genes that may lead to the cessation of feeding and/or growth and may result in the death of the insect pest. The interfering RNA molecule may confer insect resistance against the same target pest as the protein of the invention, or may target a different pest. The targeted insect plant pest may feed by chewing, sucking, or piercing. Interfering RNAs are known in the art to be useful for insect control. In other embodiments, the interfering RNA may confer resistance against a non-insect plant pest, such as a nematode pest or a virus pest.

The co-expression of more than one pesticidal agent in the same transgenic plant can be achieved by making a single recombinant vector comprising coding sequences of more than one pesticidal agent in a so called molecular stack and genetically engineering a plant to contain and express all the pesticidal agents in the transgenic plant. Such molecular stacks may be also be made by using mini-chromosomes as described, for example in U.S. Pat. No. 7,235,716. Alternatively, a transgenic plant comprising one nucleic acid encoding a first pesticidal agent can be re-transformed with a different nucleic acid encoding a second pesticidal agent and so forth. Alternatively, a plant, Parent 1, can be genetically engineered for the expression of genes of the present invention. A second plant, Parent 2, can be genetically engineered for the expression of a second pesticidal agent. By crossing Parent 1 with Parent 2, progeny plants are obtained which express all the genes introduced into Parents 1 and 2.

Transgenic plants or seed comprising a recombinant insecticidal protein of the invention can also be treated with an insecticide or insecticidal seed coating as described in U.S. Pat. Nos. 5,849,320 and 5,876,739, herein incorporated by reference. Where both the insecticide or insecticidal seed coating and the transgenic plant or seed of the invention are active against the same target insect, for example a lepidopteran target pest, the combination is useful (i) in a method for further enhancing activity of the composition of the invention against the target insect, and (ii) in a method for preventing development of resistance to the composition of the invention by providing yet another mechanism of action against the target insect. Thus, the invention provides a method of enhancing control of a lepidopteran insect population comprising providing a transgenic plant or seed of the invention and applying to the plant or the seed an insecticide or insecticidal seed coating to a transgenic plant or seed of the invention.

Even where the insecticidal seed coating is active against a different insect, the insecticidal seed coating is useful to expand the range of insect control, for example by adding an insecticidal seed coating that has activity against coleopteeran insects to a transgenic seed of the invention, which, in some embodiments, has activity against coleopteran and some lepidopteran insects, the coated transgenic seed produced controls both lepidopteran and coleopteran insect pests.

Examples of such insecticides and/or insecticidal seed coatings include, without limitation, a carbamate, a pyrethroid, an organophosphate, a friprole, a neonicotinoid, an organochloride, a nereistoxin, or a combination thereof. In another embodiment, the insecticide or insecticidal seed coating are selected from the group consisting of carbofuran, carbaryl, methomyl, bifenthrin, tefluthrin, permethrin, cyfluthrin, lambda-cyhalothrin, cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, and a combination thereof. Commercial products containing such insecticides and insecticidal seed coatings include, without limitation, Furadan® (carbofuran), Lanate® (methomyl, metomil, mesomile), Sevin® (carbaryl), Talstar® (bifenthrin), Force® (tefluthrin), Ammo® (cypermethrin), Cymbush® (cypermethrin), Delta Gold® (deltamethrin), Karate® (lambda-cyhalothrin), Ambush® (permethrin), Pounce® (permethrin), Brigade® (bifenthrin), Capture® (bifenthrin), ProShield® (tefluthrin), Warrior® (lambda-cyhalothrin), Dursban® (chlorphyrifos), Fortress® (chlorethoxyfos), Mocap® (ethoprop), Thimet® (phorate), AAstar® (phorate, flucythinate), Rampart® (phorate), Counter® (terbufos), Cygon® (dimethoate), Dicapthon, Regent® (fipronil), Cruiser® (thiamethoxam), Gaucho® (imidacloprid), Prescribe® (imidacloprid), Poncho® (clothianidin) and Aztec® (cyfluthrin, tebupirimphos).

The invention also encompasses an insecticidal composition comprising an effective insect-controlling amount of a recombinant insecticidal protein of the invention. In further embodiments, the insecticidal composition comprises a suitable agricultural carrier and an insecticidal protein of the invention. An insecticidal composition of the invention, for example a composition comprising an insecticidal protein of the invention and an agriculturally acceptable carrier, may be used in conventional agricultural methods. An agriculturally acceptable carrier is a formulation useful for applying a composition comprising a polypeptide of the invention to a plant or seed. For example, the compositions of the invention may be mixed with water and/or fertilizers and may be applied preemergence and/or postemergence to a desired locus by any means, such as airplane spray tanks, irrigation equipment, direct injection spray equipment, knapsack spray tanks, cattle dipping vats, farm equipment used in ground spraying (e.g., boom sprayers, hand sprayers), and the like. The desired locus may be soil, plants, and the like. An insecticidal composition of the invention may be applied to a seed or plant propagule in any physiological state, at any time between harvest of the seed and sowing of the seed; during or after sowing; and/or after sprouting. It is preferred that the seed or plant propagule be in a sufficiently durable state that it incurs no or minimal damage, including physical damage or biological damage, during the treatment process. A formulation may be applied to the seeds or plant propagules using conventional coating techniques and machines, such as fluidized bed techniques, the roller mill method, rotostatic seed treaters, and drum coaters.

In some embodiments, the invention encompasses a method of controlling an insect pest comprising, delivering to the insect pest or an environment thereof an effective amount of a recombinant insecticidal protein of the invention. In some aspects of these embodiments, the insecticidal protein is delivered through a transgenic plant or by topical application of an insecticidal composition comprising the chimeric insecticidal protein. In other embodiments, the recombinant insecticidal protein comprises an amino acid sequence selected from the group consisting of SEQ ID NO: 61-66 or SEQ ID NO: 115-117. In other embodiments, the insecticidal composition comprises an insecticidal Cry1Ab protein comprising SEQ ID NO: 60. In other embodiments, the transgenic plant or the insecticidal composition comprises a second insecticidal agent different from the chimeric insecticidal protein. In other embodiments, the second insecticidal agent is a protein or a dsRNA. In further embodiments, the protein is selected from the group consisting of a Cry protein, a vegetative insecticidal protein (Vip), a patatin, a protease, a protease inhibitor, a urease, an alpha-amylase inhibitor, a pore-forming protein, a lectin, an engineered antibody or antibody fragment, or a chitinase, and a combination thereof.

In some embodiments, the invention also provides a method of mitigating the impact of a transgenic corn plant expressing an insecticidal protein of the invention on non-target insects exposed to pollen from the transgenic corn plant, comprising introducing into a corn plant a DNA construct of the invention, wherein the DNA construct causes the insecticidal protein to be expressed at a lower level in pollen compared to leaf, thus mitigating the impact of the transgenic corn pollen on non-target insects exposed to the pollen.

### EXAMPLES

Embodiments of this invention can be better understood by reference to the following examples. The foregoing and following description of embodiments of the invention and the various embodiments are not intended to limit the claims, but are rather illustrative thereof. Therefore, it will be understood that the claims are not limited to the specific details of these examples. It will be appreciated by those skilled in the art that other embodiments of the invention may be practiced without departing from the spirit and the scope of the disclosure, the scope of which is defined by the appended claims. Art recognized recombinant DNA and molecular cloning techniques may be found in, for example, J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1. Identification of Endogenous Male Tissue miRNAs

Pollen-specific and tapetum-specific miRNAs were mined from the public database, “PmiRExAT” on the World Wide Web at pmirexat.nabi.res.in/index.html (Gurjar, A. K. S., et al. PmiRExAt: Plants miRNA expression atlas database and web applications. 2016. Vol. 2016: article ID baw060; doi:10.1093/database/baw060). Pollen-specific miRNAs were identified by mining the data in “File 3: 283 NR known miR of Maize versus 43 datasets Count Matrix Foldchange and Shannon Entropy,” available for download from PmiRExAT. This file contains expression data, at arm granularity, i.e. 3′-arm (3p) or 5′-arm (5p), for corn miRNAs in various tissues from publically available studies. For the invention, “pollen-specific” or “tapetum-specific” miRNAs were defined as miRNAs with 1) the highest expression in pollen or tapetum relative to other tissues and 2) low expression in non-pollen or non-tapetum tissues. Based on these criteria, three pollen-specific miRNAs were chosen for further evaluation, miRNA159h-3p, miRNA156i-3p and miRNA171i-5p. One additional pollen-preferred miRNA, miR396b-3p, was also chosen from the same dataset because it has high expression in pollen (higher than miR171i-5p and miR156i-3p), but also mid-level expression in target tissues that are fed upon by pest insects, e.g. seedling, root and silks. Three tapetum-specific miRNAs were also chosen, miR2275a-3p, miR2275b-5p and miR2275b-3p.

### Example 2. Identification of Optimal Flanking Sequences for miRNA Binding Sites

To improve miRNA accessibility to binding to an initiator sequence, flanking sequences were designed and added at the 5′ and 3′ end of each initiator sequence. The flanking sequences were designed to conform to the following: 1) to give negative free energy around the initiator sequence; 2) a size of 17 bases on the 5′ end of the initiator sequence and 13 bases on the 3′ end of the initiator sequence; and 3) low GC content. Therefore, each miRNA element of the invention comprises an initiator sequence flanked on the 5′end by 17 bases and on the 3′ end by 13 bases. The size of the miRNA element is about 51 or 52 nucleotides.

The RNAinverse tool in the ViennaRNA software package (Lorenz et al. 2011. ViennaRNA Package 2.0 Algorithms for Molecular Biology, 6:26, doi:10.1186/1748-7188-6-26) was used to identify the most optimal 17 bp 5′ and 13 bp 3′ flanking sequence for each of the DNA constructs described below. For constructs where the putative target gene sequence was used, the 17 bp 5′ and 13 bp 3′ sequences around the target binding site were provided along with the 21/22 nucleotide initiator sequence as input to RNAinverse. RNAinverse was instructed to leave the miRNA initiator sequence unchanged but to modify the endogenous flanking sequences to generate the sequence with the lowest free energy. For example, an input sequence that was 5′-tcctattcaatgataagAAGAGCACCGTTCACTCCAACtgcaaacatgtgg-3′ (SEQ ID NO: 122), was changed to an output sequence that was 5′-tcctattcaaggataagAAGAGCACCGTTCACTCCAACtgcaaacaggtgg-3′ (SEQ ID NO: 123), where the “t” at positions 11 from the 5′ end and the “t” at position 5 from the 3′ end in the input sequence were changed to “g” in the output sequence (shown as underline). For constructs where the perfect matching sequence to the miRNA was used, the most optimal flanking sequences were generated de novo. For example, an input sequence that was 5′-nnnnnnnnnnnnnnnnnCAGAGCTCCCTTCACTCCAAAnnnnnnnnnnnnn-3′ (SEQ ID N: 124), where “n” is any base, was changed to an output sequence that was 5′-cgctcacgacagcctggCAGAGCTCCCTTCACTCCAAAcattgcgcatctc-3′ (SEQ ID NO: 125).

### Example 3. Design of Constructs with miRNA Binding Sites Inserted into Insect Control Genes

Multiple constructs were made that varied, 1) the mature miRNA target or initiator sequence that was inserted into the transgene, and 2) the location of the initiator sequence in the transgene, i.e. the initiator sequence was inserted in the 5′ untranslated region (5′UTR), the 3′ untranslated region (3′UTR) or within the insecticidal protein's coding sequence (CDS).

The initiator sequences used in this example were either a perfect complementary match to the 21 or 22 nucleotide sequence of the mature miRNA (“motif”) or the initiator sequence was the same as a predicted target gene within corn plants (“tgene”), i.e. may include mismatches to the mature miRNA sequence. It is known that initiator sequences with a few mismatches to the miRNA 3′ regions, which are common in plants, are often equally effective and sometimes more effective than perfectly matched sites (Liu Q, et al. (2014). The Plant Cell. 26(2):741-53). Some corn miRNA endogenous target genes are known and have been published (Zhang L, et al. (2009) PLoS Genet. 5(11): e 1000716), but these studies do not always distinguish between the two possible mature miRNAs that can be produced from the precursor miRNA, i.e. the 3′ and 5′ arms (e.g. miR142-3p vs. miR142-5p). Therefore, the 21 or 22 nucleotide mature miRNA for each of the miRNAs described in Example 1 were obtained from miRBase and used to search for potential target genes in the MAIZE_B73_REF_5_GENOME using the software: scan_for_matches available on the World Wide Web at “blog.theseed.org/servers/2010/07/scan-for-matches.html, using the rules disclosed by Schwab et al. 2005. Developmental Cell 8:517-527. Briefly, the rules are: 1) only one mismatch allowed between positions 2 to 12 inclusive; 2) no mismatches allowed at positions 10 and 11; 3) no more than 2 consecutive mismatches allowed after position 12; and 4) no more than 3 total mismatches (excluding mismatch at position 1) across the length of the mature miRNA. A list of computationally predicted target genes was generated for each miRNA.

Additional basic analyses (e.g. pairwise sequence alignment) of the scan_for_matches output provided the location of the binding site for the miRNA in each predicted target gene (e.g. exon, UTR and so forth), the number of mismatches between the miRNA and the binding site sequence, and the location(s) of the mismatches relative to the miRNA sequence. Gene expression data from a gene atlas expression study of a proprietary maize line and protein expression data from a public study (PMID: 27540173) were also collected to identify putative target genes with the expected expression pattern for a true endogenous target gene (i.e. expression repressed in pollen compared to other tissues)

Lastly, the binding sequences in the predicted target genes were compared to the sequences of closely related but non-pollen-specific miRNAs or non-tapetum-specific miRNAs to avoid inserting sequences that could potentially be bound by non-pollen/non-tapetum specific miRNAs which may inadvertently down-regulate the engineered insect control transgene in maize tissues where expression is still required for control of pest insects, e.g. leaf tissue. An example of the data generated for one of the mature miRNA sequences, miR159h-3p, is shown in Table 1.

TABLE 1
Sample data set used for choosing male tissue-specific/tissue-preferred miRNAs.

| | | | | Gene | |
|---|---|---|---|---|---|
| | | No. | Mismatch | Expression | Other miRNAs |
| B73_V5 Gene ID | Target Site Location | Mismatches | Position | in Pollen | Targeting Gene |
| Zm00001d003864 | 5’UTR | 2 | 6, 15 | M | mir159a/b/j/k/f-3p |
| Zm00001d043131 | Exon | 3 | 1, 20, 21 | M/H | mir159a/b/j/k/f-3p |
| Zm00001d049606 | Exon/5’UTR | 4 | 1, 12, 15, 21 | L/M | None |
| Zm00001d010309 | 5’UTR/intron/ 3'UTR | 3 | 8, 14, 19 | H | mir159a/b/j/k/f-3p |
| Zm00001d046517 | Exon | 3 | 7, 20, 21 | L | mir159a/b/j/k/f-3p |
| Zm00001d046518 | Exon | 3 | 7, 20, 21 | L | mir159a/b/j/k/f-3p |
| Zm00001d046519 | Exon | 3 | 7, 20, 21 | L | mir159a/b/j/k/f-3p |
| Zm00001d029563 | 5’UTR/intron | 4 | 1, 8, 14, 16 | H | None |

Based on the type of data shown in Table 1 for all four pollen miRNAs described in Example 1, twelve (12) constructs were made using a cry1Ab coding sequence (SEQ ID NO: 67) as the insecticidal transgene. The 12 constructs are described in Table 2. Construct 24366 is the control construct comprising a full-length cry1Ab-17 coding sequence without any miRNA element. The expression of Cry1Ab is driven by a maize ubiquitin promoter and NOS terminator. 23466 also contains a PMI selectable marker expression cassette for corn transformation. In addition, ten (10) constructs were made using a vip3 coding sequence (SEQ ID NO: 118) as the insecticidal transgene and miR2275. miR2275 is expressed preferentially in the tapetum layer within the anther and pre-pollen meiocytes. The presence of miR2275 is intended to provide anther-specific down-regulation of Vip3A in transgenic maize plants. The 10 constructs are described in Table 3. Construct 23705 is the control construct comprising a full-length vip3A coding sequence without any miRNA element. The expression of Vip3 is driven by a maize ubiquitin promoter and 35S terminator. 23705 also contains a PMI selectable marker expression cassette for corn transformation

TABLE 2
CrylAb miRNA constructs.

| | | | | Initiator | |
|---|---|---|---|---|---|
| | Construct | | Initiator | Sequence | Location in |
| | SEQ | | Sequence | SEQ | crylAb |
| Construct | ID NO: | miRNA-arm | Type | ID NO: | gene |
| 24366 | 47 | control | na | na | na |
| 24372 | 57 | miR159h-3p | motif | 29 | CDS |
| 24373 | 48 | miR159h-3p | tgene | 30 | CDS |
| 24374 | 49 | miR156i-3p | motif | 31 | CDS |
| 24375 | 50 | miR171i-5p | motif | 32 | CDS |
| 24376 | 51 | miR396b-3p | motif | 33 | CDS |
| 24377 | 52 | miR396b-3p | tgene | 34 | CDS |
| 24378 | 58 | miR159h-3p | motif | 29 | 5’UTR |
| 24379 | 53 | miR159h-3p | tgene | 30 | 5’UTR |
| 24380 | 54 | miR156i-3p | motif | 31 | 5’UTR |
| 24381 | 55 | miR171i-5p | motif | 32 | 5’UTR |
| 24382 | 59 | miR396b-3p | motif | 33 | 5’UTR |
| 24383 | 56 | miR396b-3p | tgene | 34 | 5’UTR |

TABLE 3
Vip3 miRNA constructs.

| | | | | Initiator | |
|---|---|---|---|---|---|
| | Construct | | Initiator | Sequence | Location |
| | SEQ | | Sequence | SEQ : | in |
| Construct | ID NO: | miRNA-arm | Type | ID NO | Vip3 gene |
| 23705 | 104 | Control | na | na | na |
| 23708 | 105 | miR2275a-3p | motif | 94 | 3’UTR |
| 23711 | 106 | miR2275b-5p | motif | 95 | 3’UTR |
| 23712 | 107 | miR2275b-3p | motif | 96 | 3’UTR |
| 23713 | 108 | miR2275b-3p | motif | 96 | 5’UTR |
| 23714 | 109 | miR2275b-3p | motif | 96 | 3’UTR |
| 23715 | 110 | miR2275b-3p | tgene | 97 | 3’UTR |
| 23716 | 111 | miR2275b-3p | motif | 96 | CDS |
| 23717 | 112 | miR2275b-3p | motif | 96 | CDS |
| 23718 | 113 | miR2275b-3p | motif | 96 | CDS |

### Example 4. Transient Transformation

This example describes testing the constructs designed in Example 3 in transient maize assays to determine whether any maize endogenous miRNAs with similar binding sequences to the selected miRNAs of the invention but with different tissue location could target the engineered transgene in the constructs for down regulation in tissues other than male reproductive tissue, e.g. leaf. An example of such a maize transient assay is disclosed in U.S. Pat. No. 8,642,839, herein incorporated by reference. The skilled person will recognize that variations in and modifications to the transient assay system may be made to achieve the same intended result. Constructs described in Example 3 were tested in the maize leaf transient assay. For the Cry1Ab constructs, a construct comprising the full-length cry1Ab coding sequence (24366) without the miRNA element was used as a positive control. An empty vector (EHA101) was used as the negative control. A third control construct (18515) that comprises a coral fluorescent protein (CFP) was also tested in each assay. Expression levels of the Cry1Ab protein were normalized against the expression level of CFP. Results are shown in Table 4.

TABLE 4
Results of a transient maize assay for CrylAb-miRNA constructs.

| | | CrylAb | CrylAb |
|---|---|---|---|
| | | Conc ± 0SE | Conc ± 0SE |
| | | (ng CrylAb/ | (ng CrylAb/ |
| Construct | Expression Cassette | mg TSP) | μg CFP) |
| 24366 | Positive Control | 9.0 ± 01.5 | 240.28 ± 0118.6 |
| 24372 | miR159h-motif-CDS | 1.8 ± 00.2 | 40.91 ± 082.61 |
| 24373 | miR159h-gene-CDS | 8.0 ± 02.0 | 204.90 ± 078.93 |
| 24374 | miR156i-motif-CDS | 8.0 ± 01.2 | 190.02 ± 0100.66 |
| 24375 | miR171i-motif-CDS | 5.2 ± 00.9 | 141.70 ± 019.58 |
| 24376 | miR396b-motif-CDS | 7.9 ± 01.3 | 200.74 ± 083.30 |
| 24377 | miR396b-gene-CDS | 5.0 ± 01.5 | 103.25 ± 070.07 |
| 24378 | miR159h-motif-5’UTR | 4.2 ± 00.8 | 89.79 ± 090.31 |
| 24379 | miR159h-gene-5’UTR | 11 ± 02.6 | 257.79 ± 0104.33 |
| 24380 | miR156i-motif-5’UTR | 12.8 ± 03.2 | 323.10 ± 061.72 |
| 24381 | miR171i-motif-5’UTR | 11.2 ± 01.8 | 284.45 ± 070.02 |
| 24382 | miR396b-motif-5’UTR | 4.2 ± 00.8 | 114.88 ± 044.16 |
| 24383 | miR396b-gene-5’UTR | 6.9 ± 01.1 | 177.20 ± 053.40 |
| EHA101 | Negative Control | 0.0 ± 00.0 | 0.0 ± 00.0 |

Expression of Cry1Ab protein was detected for all of the Cry1Ab-miRNA constructs, although expression for three of the constructs was significantly less than the control. Based on the transient expression results the following constructs were chosen for testing in stable maize transformation experiments: 24373, 24374, 24375, 24376, 24377, 24379, 24380 and 24381, as well as the Cry1Ab control, 24366.

Expression of Vip3A protein was detected for all of the Vip3A-miRNA constructs, although expression for four of the constructs was significantly less than the control. All of the Vip3A-miR2275 constructs were tested in stable transformation experiments.

### Example 5. Stable Transformation of Maize

This example describes testing the constructs selected in Example 4 in stable maize transformation experiments.

Each recombinant vector described above comprises two expression cassettes. The first expression cassette comprises an insecticidal protein-coding sequence operably linked to a DNA sequence encoding a microRNA (miRNA) element that is male tissue-specific or male tissue-preferred in a corn plant. The first expression cassette further comprises a maize ubiquitin promoter and NOS terminator both of which are operably linked to the coding-sequence/miRNA element combination. The second cassette comprises a maize Ubi1 promoter operably linked to a pmi coding sequence that encodes the selectable marker phosphomannose isomerase (PMI), which is operably linked to a maize Ubi1 terminator.

A recombinant vector of the invention is transformed into Agrobacterium tumefaciens using standard molecular biology techniques. To prepare the Agrobacteria for transformation, cells are cultured in liquid YPC media at 28° C. and 220 rpm overnight.

Agrobacterium transformation of immature maize embryos is performed essentially as described in Negrotto et al., 2000, Plant Cell Reports 19: 798-803. For this example, all media constituents are essentially as described in Negrotto et al., supra. However, various media constituents known in the art may be substituted.

Briefly, Agrobacterium strain LBA4404 (pSB1) containing the binary vector plant transformation vector was grown on YEP (yeast extract (5 g/L), peptone (10 g/L), NaCl (5 g/L), 15 g/1 agar, pH 6.8) solid medium for 2-4 days at 28° C. Approximately 0.8×10⁹ Agrobacterium are suspended in LS-inf media supplemented with 100 μM As (Negrotto et al., supra). Bacteria were pre-induced in this medium for approximately 30-60 minutes.

Immature embryos from a suitable corn genotype were excised from 8-12 day old ears into liquid LS-inf+100 μM As. Embryos were rinsed once with fresh infection medium. Agrobacterium solution was then added and embryos were vortexed for 30 seconds and allowed to settle with the bacteria for about 5 minutes. The embryos were then transferred scutellum side up to LSAs medium and cultured in the dark for two to three days. Subsequently, between 20 and 25 embryos per petri plate were transferred to LSDc medium supplemented with cefotaxime (250 mg/1) and silver nitrate (1.6 mg/1) and cultured in the dark at 28° C. for 10 days.

Immature embryos, producing embryogenic callus were transferred to LSD1M0.5S medium. The cultures were selected on this medium for about 6 weeks with a subculture step at about 3 weeks. Surviving calli were transferred to Reg1 medium supplemented with mannose. Following culturing in the light (16 hour light/8 hour dark regiment), green tissues are then transferred to Reg2 medium without growth regulators and incubated for about 1-2 weeks. Plantlets were transferred to Magenta GA-7 boxes (Magenta Corp, Chicago Ill.) containing Reg3 medium and grown in the light.

Following transformation, selection, and regeneration, T0 plants were assayed for the presence of the pmi gene and the cry1Ab or vip3A coding sequence using TaqMan® analysis. Plants were also tested for the presence of the vector backbone. Transgenic maize plants negative for the vector backbone and comprising one copy of the transgene from the recombinant vector were transferred to a greenhouse and tested for insecticidal activity against a lepidopteran insect pest, for example, European corn borer and fall armyworm.

### Example 6. Expression Level and Pattern of Expression of Cry1Ab

This example describes T0 plant analysis to determine the level of expression of Cry1Ab protein in T0 transgenic corn plants stably transformed with the Cry1Ab-miRNA constructs described in Example 3.

Growth stages of corn are divided into vegetative stages (V) and reproductive stages (R). Subdivisions of the V stages are designated numerically as V1, V2, V3, and so forth through V(n), where (n) represents the last leaf stage before tasseling (VT). The reproductive stages are designated numerically as R1=silking through R6=maturity.

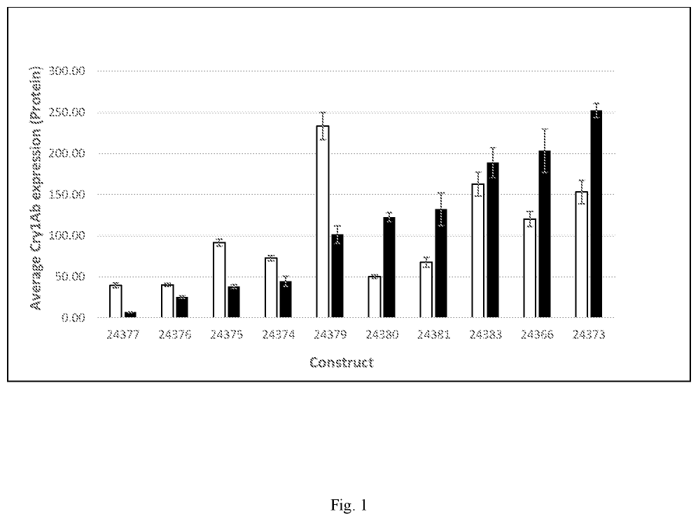

Transgenic corn plants selected in Example 5 were grown to the R1 stage under controlled conditions in a greenhouse. At the R1 stage, samples were taken from leaf, pollen and silks from multiple T0 plants for each construct and expression levels of the insect control transgene and protein were measured using qRT-PCR and ELISA, respectively. In addition, insect bioassays were performed on leaf and silk tissue to ensure that that the insecticidal protein encoded by the engineered transgene retained toxicity to a lepidopteran insect pest. Results of the ELISA assays are shown in Table 5 and FIG. 1.

Results demonstrate that corn plants comprising recombinant DNA constructs comprising an insecticidal protein-coding sequence operably linked to a DNA sequence encoding a microRNA (miRNA) element that is male tissue-specific or male tissue-preferred have lower expression of the insecticidal protein in male reproductive tissue compared to leaf tissue. Constructs 24374, 24375, 24376, 24377 and 24379 had significantly lower levels of Cry1Ab in pollen compared to leaf. All of these constructs, except 24379, have the miRNA element within the Cry1Ab coding sequence. Surprisingly, constructs comprising the miRNA element in the 5′UTR, except 24379, had significantly higher levels of Cry1Ab in the pollen compared to leaf

TABLE 5
Expression levels of Cry lAb protein in TO transgenic corn plants.

| | Insecticidal Protein | Cry 1Ab Concentration (ng/mg TSP) | |
|---|---|---|---|
| Construct | Coding-Sequence | Mean Leaf (±SE) | Mean Pollen (±SE) |
| 24366 | CrylAb-Control | 120.18 ± 09.10 | 203.17 ± 026.61 |
| 24377 | miR396b/tgene/CDS | 39.40 ± 02.93 | 6.81 ± 00.49 |
| 24376 | miR396b/motif/CDS | 40.15 ± 01.88 | 25.28 ± 01.44 |
| 24383 | miR396b/tgene/5’UTR | 162.81 ± 014.54 | 188.87 ± 018.31 |
| 24380 | miR156i/motif/5’UTR | 50.13 ± 02.10 | 122.56 ± 05.68 |
| 24374 | miR156i/motif/CDS | 72.57 ± 03.26 | 44.44 ± 06.20 |
| 24375 | miR171i/motif/CDS | 91.46 ± 04.20 | 38.06 ± 02.45 |
| 24381 | miR171i/motif/5’UTR | 67.57 ± 05.87 | 132.10 ± 020.09 |
| 24373 | miR159h/tgene/CDS | 153.15 ± 014.33 | 252.35 ± 08.83 |
| 24379 | miR159h/tgene/5’UTR | 233.45 ± 016.57 | 101.37 ± 010.69 |

Leaf and silk tissue from multiple T0 plants at the R1 stage were tested for insecticidal activity against European corn borer (ECB; Ostrinia nubilalis) and corn earworm (CEW; Helicoverpa zea), respectively, using tissue excision bioassays. Briefly, plant tissue (leaf and/or silks) is excised from an individual plant and placed in a sealable container. Each tissue sample is infested with neonate larvae of a lepidopteran target pest, then incubated at room temperature for about 5 days.

Results of the bioassays, shown in Table 6, demonstrate that the Cry1Ab-miRNA constructs of the invention express at high enough levels in leaf and silk to control the intended target pest, with the exception of the 24377 construct. Although 100% of the T0 plants tested that comprised the 24377 construct produced 100% mortality to ECB in a leaf bioassay, none of the T0 plants produced 100% mortality to CEW in silk bioassays.

TABLE 6
Results of insect bioassays of tissue from
TO plants comprising DNA constructs.

| | | % of T0 plants with 100% | |
|---|---|---|---|
| | Insecticidal | insect mortality | |
| | Protein Coding- | ECB Leaf | CEW Silk |
| Construct | Sequence | Bioassay | Bioassay |
| 24366 | CrylAb-Control | 100 | 68.4 |
| 24377 | miR396b/tgene/CDS | 100 | 0.0 |
| 24376 | miR396b/motif/CDS | 100 | 27.3 |
| 24383 | miR396b/tgene/5'UTR | 91.3 | 95.2 |
| 24380 | miR156i/motif/5'UTR | 95.8 | 21.05 |
| 24374 | miR156i/motif/CDS | 100 | 80.0 |
| 24375 | miR171i/motif/CDS | 100 | 95.4 |
| 24381 | miR171i/motif/5'UTR | 87.5 | 72.7 |
| 24373 | miR159h/tgene/CDS | 92.3 | 75.0 |
| 24379 | miR159h/tgene/5'UTR | 100 | 95.2 |

### Example 7. Expression Level and Pattern of Expression of Vip3A

This example describes T0 plant analysis to determine the level of expression of Vip3A protein in T0 transgenic corn plants stably transformed with the Vip3A-miRNA constructs described in Example 3.

Transgenic corn plants selected in Example 5 were grown to the R1 stage under controlled conditions in a greenhouse. At the R1 stage, samples were taken from leaf, pollen and silks from multiple T0 events for each construct and expression levels of the insect control transgene and protein were measured using qRT-PCR and ELISA, respectively. In addition, insect bioassays were performed on leaf (fall armyworm; FAW) and silk tissue (corn earworm; CEW) to ensure that the Vip3 protein encoded by the engineered transgene retained toxicity to a lepidopteran insect pest. Results of the ELISA assays and bioassays are shown in Table 7. Leaf and silk bioassay results are shown as “++,” “+,” or “−” indicating the percentage of T0 events that had insecticidal leaves and/or silks, where “++”=≥50% of the events, “+”=<50% of the events and “−”=0% of the events.

TABLE 7
Vip3A expression levels and bioassay results of T0 events.

| | | | | | Avg % Reduction | Leaves | Silks |
|---|---|---|---|---|---|---|---|
| | Avg. Vip3A Protein Level | | | | Anther vs. Leaf (vs. | Toxic to | Toxic to |
| Construct | Leaf | Anther | Pollen | Silk | 23705 Control) | FAW | CEW |
| 23705 | 1172.53 | 498.17 | 129.00 | 431.95 | 0.00 | + | + |
| 23712 | 1287.99 | 180.63 | 166.58 | 398.22 | 63.74 | ++ | ++ |
| 23708 | 875.32 | 101.36 | 242.03 | 231.98 | 79.65 | ++ | ++ |
| 23711 | 940.86 | 676.72 | 352.70 | 550.10 | −35.84 | ++ | ++ |
| 23713 | 275.94 | 54.05 | 31.33 | 64.18 | 89.15 | ++ | − |
| 23715 | 664.40 | 279.15 | 181.72 | 98.48 | 43.97 | ++ | ++ |
| 23714 | 866.44 | 173.89 | 174.28 | 243.80 | 65.09 | ++ | ++ |
| 23718 | 201.97 | 71.51 | 12.19 | 120.68 | 85.64 | + | − |
| 23716 | 265.74 | 24.51 | 14.69 | 80.52 | 95.08 | − | − |
| 23717 | 174.26 | 24.66 | 29.50 | 164.77 | 95.05 | + | − |

Results demonstrate that transgenic corn events (plants) comprising recombinant DNA constructs comprising a Vip3 insecticidal protein-coding sequence operably linked to a DNA sequence encoding a microRNA (miRNA) element that is male tissue-specific or male tissue-preferred have lower expression of the Vip3 protein in male reproductive tissue compared to leaf tissue. When compared to the positive control, constructs 23712, 23708, 23713, 23714, 23718, 23716 and 23717 had significantly lower levels of Vip3 in anther, the tissue where the miR2275 RNA is active, compared to leaf. Several of the constructs not only had lower Vip3 levels in anthers but also had lower levels of Vip3 protein in tissues where the miR2275 is not active. Such constructs still have utility if the lower Vip3 levels in other tissues does not go below the insecticidal level. This was the case for constructs, 23708, 23714 and 23715 where the Vip3 level in leaf and silks was lower than the control but yet leaf tissue and silk tissue from plant comprising these constructs was still insecticidal to a target pest. Construct 23713 had lower Vip3 protein levels in leaf and silk tissue compared to the positive control but only the leaf tissue from transgenic events comprising that construct were insecticidal to a target pest. All of the constructs having the miR2275 element within the Vip3 coding sequence produced plants that had lower Vip3 levels in all tissues analyzed. However, transgenic events comprising two of these constructs, 23718 and 23717, had leaves that were insecticidal to a target pest. The constructs with the highest reduction of Vip3 protein in anthers while maintaining insecticidal activity in both leave and silks were 23708, 23714 and 23712. All three of these constructs comprise the 3p arm of miR2275, have a motif initiator sequence type, and have the miR2275 initiator sequence located in the 3′ UTR of the Vip3 gene.

Next generation T1 plants from transgenic T0 events comprising constructs 23705, 23712, 23708, 23713 and 23717 were tested and analyzed as described above. The selectable marker construct, comprising a phosphomannose isomerase (PMI) gene, and not comprising a miR2275 initiator sequence, was used as a negative control. Results of the T1 plant analysis are shown in Table 8.

TABLE 8
Vip3 expression levels and bioassay results of T1 plants.

| | Avg. Vip3A | | Avg % Reduction | Leaves | Silks | Avg. PMI Protein | |
|---|---|---|---|---|---|---|---|
| | Protein Level | | Anther vs. Leaf (vs. | Toxic to | Toxic to | Level | |
| Construct | Leaf | Anther | 23705 Control) | FAW | CEW | Leaf | Anther |
| 23705 | 358.79 | 373.01 | 0.00 | ++ | + | 43.50 | 17.33 |
| 23708 | 425.44 | 91.59 | 75.44 | ++ | + | 49.11 | 19.71 |
| 23712 | 326.87 | 116.00 | 68.90 | ++ | + | 46.59 | 23.10 |

As with T0 events, T1 plants comprising constructs 23708 and 23712 had significantly lower Vip3 protein level in anthers compared to the positive control, due to the presence of the miR2275 initiator sequence and the endogenous activity of the miR2275 microRNA. The PMI expression cassette, that did not comprise a microRNA initiator sequence, expressed equal levels of PMI protein across all construct treatments and tissues. These results demonstrate that both miR2275a and miR2275b can recognize their respective initiator sequences in planta to down regulate the expression of an insecticidal protein like Vip3 in male reproductive tissue or corn.

### Example 8. Genome Editing in Plant Cells In Situ to Generate Modified Chimeric Proteins

This example illustrates the use of genome editing of a plant cell genome in situ to incorporate mutations, to make a DNA construct described herein (including but not limited to a DNA construct described in Examples 3) by incorporating a miRNA element into a coding sequence for a wild-type Cry1Ab or Vip3 insecticidal protein or into a coding sequence for an already modified Cry1Ab or Vip3 protein.

Targeted genome modification, also known as genome editing, is useful for introducing mutations in specific DNA sequences. These genome editing technologies, which include zinc finger nucleases (ZNFs), transcription activator-like effector nucleases (TALENS), meganucleases and clustered regularly interspaced short palindromic repeats (CRISPR) have been successfully applied to over 50 different organisms including crop plants. See, e.g., Belhaj, K., et al., Plant Methods 9, 39 (2013); Jiang, W., et al., Nucleic Acids Res, 41, e188 (2013)). The CRISPR/Cas system for genome editing is based on transient expression of Cas9 nuclease and an engineered single guide RNA (sgRNA) that specifies the targeted polynucleotide sequence.

Cas9 is a large monomeric DNA nuclease guided to a DNA target sequence with the aid of a complex of two 20-nucleotide (nt) non-coding RNAs: CRIPSR RNA (crRNA) and trans-activating crRNA (tracrRNA), which are functionally available as single synthetic RNA chimera. The Cas9 protein contains two nuclease domains homologous to RuvC and HNH nucleases. The HNH nuclease domain cleaves the complementary DNA strand, whereas the RuvC-like domain cleaves the non-complementary strand and, as a result, a blunt cut is introduced in the target DNA.

When the Cas9 and the sgRNA are transiently expressed in living maize cells, double strand breaks (DSBs) in the specific targeted DNA is created in the transgenic maize cell. Mutation at the break site is introduced through the non-homologous end joining and homology-directed DNA repair pathways.

Specific mutations are introduced into a cry1Ab coding sequence, for example SEQ ID NO: 67, or a vip3 coding sequence, for example SEQ ID NO: 118, or a modified cry1Ab and/or vip3 coding sequence, through the use of recombinant plasmids expressing the Cas9 nuclease and the sgRNA target that is maize codon optimized for the cry1Ab or vip3 or modified cry1Ab or vip3 sequence in the transgenic maize. Implementation of the method is by an agroinfiltration method with Agrobacterium tumefaciens carrying the binary plasmid harboring the specified target sequence of interest. After the sgRNA binds to the target insecticidal protein coding sequence, the Cas9 nuclease makes specific cuts into the coding sequence and introduces the desired mutation(s) during DNA repair, for example introducing a miRNA element into the cry1Ab coding sequence. Thus, a now mutated cry1Ab insecticidal protein coding sequence will encode a Cry1Ab-miRNA insecticidal protein.

Plant cells comprising the genome edited cry1Ab-miRNA insecticidal protein coding sequences are screened by PCR and sequencing. Calli that harbor genome edited cry1Ab-miRNA insecticidal protein coding sequences are induced to regenerate plants for phenotype evaluation for insecticidal activity of the expressed insecticidal protein against a lepidopteran pest insect and tested for reduced expression in the pollen as compared to leaf tissue.

It should be understood that the examples and embodiments described herein are for illustrative purposes only and that various modifications or changes in light thereof of the description will be suggested to persons skilled in the art and are to be included within the spirit and purview of this application and the scope of the appended claims.

All publications and patent applications mentioned in this specification are indicative of the level of skill of those skilled in the art that this invention pertains. All publications and patent applications are herein incorporated by reference to the same extent as if each individual publication or patent application was specifically and individually indicated to be incorporated by reference.

1. A recombinant DNA construct comprising an insecticidal protein-coding sequence operably linked to a DNA sequence encoding a microRNA (miRNA) element that is male tissue-specific or male tissue-preferred in a corn plant, wherein said miRNA element is heterologous with respect to said insecticidal protein-coding sequence.

2. The recombinant DNA construct of claim 1, wherein said miRNA element is comprised within a 5′ untranslated region of said insecticidal protein-coding sequence, or within a 3′ untranslated region of said insecticidal protein-coding sequence, or within the insecticidal protein-coding sequence.

3. The recombinant DNA construct of claim 1, wherein said male tissue is pollen and/or tapetum.

4. The recombinant DNA construct of claim 1, wherein said miRNA element comprises a sequence that encodes at least one miRNA initiator sequence.

5. The recombinant DNA construct of claim 4, wherein said miRNA initiator sequence is selected from the group consisting of SEQ ID NO: 29-34 or SEQ ID NO: 94-97.

6. The recombinant DNA construct of claim 4, wherein said sequence that encodes said miRNA initiator sequence is selected from the group consisting of SEQ ID NO:41-46 or SEQ ID NO: 98-101.

7.-8. (canceled)

9. The recombinant DNA construct of claim 4, wherein said miRNA element is selected from the group consisting of SEQ ID NO: 10-18 or SEQ ID NO: 74-78.

10.-16. (canceled)

17. A recombinant vector comprising the DNA construct of claim 1.

18. (canceled)

19. A transgenic corn plant comprising the DNA construct of claim 1.

20. A seed, progeny, or plant part of the transgenic corn plant of claim 19, wherein said seed, progeny, or plant part comprises the DNA construct.

21. A method of making a DNA construct comprising identifying an endogenous male tissue-specific or male tissue-preferred corn microRNA (miRNA), constructing an miRNA element that encodes at least one miRNA initiator sequence that is recognized by said corn miRNA and operably linking said miRNA element to an insecticidal protein-coding sequence.

22.-25. (canceled)

26. A method of selectively reducing the expression of a recombinant insecticidal protein in a male reproductive tissue of a transgenic corn plant comprising expressing in said transgenic corn plant a chimeric gene comprising an insecticidal protein-coding sequence encoding said recombinant insecticidal protein and at least a first male tissue-specific miRNA element or a male tissue-preferred miRNA element operably linked to said insecticidal protein-coding sequence, wherein said miRNA element encodes at least one miRNA initiator sequence.

27. The method of claim 26, wherein said miRNA element is comprised within a 5′ untranslated region of said insecticidal protein-coding sequence, or within a 3′ untranslated region of said insecticidal protein-coding sequence or within the insecticidal protein-coding sequence.

28. The method of claim 26, wherein the miRNA element comprises at least two miRNA initiator sequences.

29. The method of claim 26, wherein the wherein said male tissue is pollen and/or tapetum.

30-69. (canceled)

70. A method of mitigating the impact of a transgenic corn plant expressing an insecticidal protein on non-target insects exposed to pollen from said transgenic corn plant, comprising introducing into a corn plant the DNA construct of claim 1, wherein the DNA construct causes the insecticidal protein to be expressed at a lower level in pollen compared to leaf, thus mitigating the impact of the transgenic corn pollen on non-target insects exposed to said pollen.
